(19) United States
(12) Patent Application Publication
(10) Pub. No.: US 2014/0017330 A1
(43) Pub. Date: Jan. 16, 2014

(54) Activated Nucleoside Analog Conjugates and Methods of Use Thereof

(71) Applicant: Serguei V. Vinogradov, Omaha, NE (US)

(72) Inventor: Serguei V. Vinogradov, Omaha, NE (US)

(73) Assignee: BOARD OF REGENTS OF THE UNIVERSITY OF NEBRASKA, Lincoln, NE (US)

(21) Appl. No.: 14/002,510

(22) PCT Filed: Apr. 4, 2012
(86) PCT No.: PCT/US2012/032129

§ 371 Date: Sep. 11, 2013

Related U.S. Application Data
(60) Provisional application No. 61/471,288, filed on Apr. 4, 2011.

(51) Int. Cl. A61K 9/14; A61K 45/06

(52) U.S. Cl. CPC A61K 9/146; A61K 45/06
USPC 424/499; 536/123.1; 424/501; 424/78.26; 424/78.29

(57) ABSTRACT

The present invention provides nanogel formulations and methods of use thereof.

This application claims priority under 35 U.S.C. §119(e) to U.S. Provisional Patent Application No. 61/471,288, filed Apr. 4, 2011. The foregoing application is incorporated by reference herein.

This invention was made with government support under Grant No. CA136921 awarded by the National Institutes of Health. The government has certain rights in the invention.

### FIELD OF THE INVENTION

The present invention relates generally to drug delivery systems. More specifically, the present invention relates to compositions and methods for the delivery of cytotoxic nucleoside analogs.

### BACKGROUND OF THE INVENTION

Despite several decades of research and drug development, progress in the clinical struggle against cancer has been only moderate. Development of resistance by cancer cells to chemotherapeutic agents has currently become a major clinical problem, limiting the effectiveness of the treatment of hematological malignancies as well as solid tumors. Multiple studies have helped to identify many mechanisms of drug resistance, but in general terms, they can all be reduced to the prevention of a drug from entering cells, deactivation of drug molecules, or the enhanced resistance of cancer cells to apoptosis. The first group includes deficiencies in membrane nucleoside transporters or the overexpression of ATP-dependent drug efflux transporters like P-glycoprotein (MRP, BCRP) and other membrane proteins (MRP) responsible for drug efflux. In the second group, the drug metabolism/degradation and reduced levels of enzymatic drug activation should be mentioned. In the third group, the induction of antiapoptotic mechanisms, as well as the suppression of proapoptotic pathways, plays important roles in drug resistance (Johnstone et al. (2002) Cell 108:153-164). Accordingly, there is a strong need for superior chemotherapeutic agent formulations that overcome cancer cell resistance.

### SUMMARY OF THE INVENTION

In accordance with the instant invention, nanogels (nanoparticles) for the delivery of therapeutic agents are provided. In a particular embodiment, the nanoparticles comprise at least one hydrophilic polymer conjugated to hydrophobic moieties and at least one therapeutic agent conjugated to the hydrophilic polymer by a polyphosphate linkage (e.g., a tetraphosphate). The hydrophobic moieties form a hydrophobic core of the nanoparticle which is encompassed by the hydrophilic polymer. In a particular embodiment, the therapeutic agent is a nucleoside analog. In a particular embodiment, the hydrophilic polymer comprises hydroxyl groups. The nanoparticles may further comprise at least one targeting ligand. The nanoparticles may also further encapsulate at least one therapeutic agent, particularly a hydrophobic therapeutic agent. Compositions comprising at least one nanoparticle of the instant invention and at least one pharmaceutically acceptable carrier are also provided.

In accordance with another aspect of the instant invention, methods for treating, inhibiting, and or preventing a disease or disorder in a subject are provided. The methods comprise administering at least one nanoparticle of the instant invention, optionally in a composition with at least one pharmaceutically acceptable carrier, to the subject. In a particular embodiment, the disease or disorder is cancer or a viral infection.

In accordance with another aspect of the instant invention, methods of synthesizing the nanoparticle of the instant invention are also provided.

### BRIEF DESCRIPTIONS OF THE DRAWING

FIG. 1 provides a schematic of the formation of compact nanogels from polymer drug conjugates.

FIG. 2A provides a schematic of the synthetic steps in the preparation of CPVA conjugates. FIG. 2B provides a schematic of the synthesis of CDex-conjugates.

FIG. 3 provides ¹H-NMR spectrum of CPVA31 in D₂O (FIG. 3A) and in DMSO-d₆ (FIG. 3B). Crossed peaks correspond to residual solvent peaks. Values on the x-axis are in ppm.

FIG. 4 provides ¹H-NMR spectra of CDex9 in D₂O (FIG. 4A) and in DMSO-d₆ (FIG. 4B). Crossed peaks correspond to residual solvent peaks. Values on the x-axis are in ppm.

FIG. 5 provides the ³¹P NMR spectrum of polymeric conjugates, CPVA31-p₄FdU (FIG. 5A) and CDex9-p₄FdU (FIG. 5B). Phosphorus signals are designated a-e.

FIG. 6 provides transmission electron microscopy (TEM) images of nanogels formed from polymer conjugates: CPVA31-p₄FdU (FIG. 6A), its spermine complex (Spe; FIG. 6B), CDex9-p₄FdU (FIG. 6C), and its spermine complex (FIG. 6D). Samples were stained with vanadate.

FIG. 7 shows the enzymatic hydrolysis of polymeric conjugates by snake venom phosphodiesterase I (VPDE). The enzyme hydrolyzes the P—O bond at α-phosphate group in nanogel conjugate resulting in nucleoside 5′-phosphate (2) (FIG. 7A). FIG. 7B provides ion-pair HPLC profiles of initial (a) and hydrolyzed (b) CPVA31-p₄FdU. FIG. 7C provides ion-pair HPLC profiles of initial (a) and hydrolyzed (b) CDex9-p₄T after 24 hour incubation with 0.01 units of VPDE enzyme.

FIG. 8 shows the in vitro drug release from polymeric conjugates CPVA31-p₄FdU (FIG. 8A) and CDex9-p₄FdU (FIG. 8B) at different pH in buffered saline.

FIG. 9 provides a schematic of the mechanism of acidic hydrolysis of polymeric conjugates and release of phosphorylated nucleosides (R=cholesterol).

FIG. 10 provides graphs of the cytotoxicity of polymeric conjugates in drug-resistant human T-lymphoma CEM/araC/8 cells (FIGS. 10A-10C) and prostate carcinoma PC-3 cells (FIG. 10D).

FIG. 11 provides graphs showing the tumor growth inhibition in mice with subcutaneous human prostate carcinoma PC-3 (FIG. 11A) and gemcitabine-resistant follicular lymphoma RL7/G (FIG. 11B) tumors following the peritumoral injections of the polymeric conjugate CPVA31-p₄FdU (dose 80 mg/kg or 10 mg FdU/kg). The data were statistically significant with P<0.05 between (a) control and (b) treatment groups. FIG. 11C provides tumor photographs in the end of the experiment B taken from control and treatment groups.

FIG. 12 provides IR spectrum of PVA31 polymer (a), CPVA31 (b) and CPVA31-p4FdU conjugates (c).

FIG. 13 provides a schematic of the formation of dual drug nanoformulations.

FIG. 14 provides graphs of the cytotoxicity of dual drug nanoformulations in pancreatic cancer cells (FIGS. 14A and 14C) and breast cancer cells (FIGS. 14B and 14D). FIG. 14A compares the cytotoxicity of paclitaxel (Pcl) versus the dual drug nanoformulation of CPVAp4FdU and paclitaxel (NG). FIG. 14B compares the cytotoxicity of 17-AAG versus the dual drug nanoformulation of CPVAp4FdU and 17-AAG. FIG. 14C compares the cytotoxicity of gemcitabine (Gen) versus the dual drug nanoformulation of CPVAp4FdU and gemcitabine (NG-GemC). FIG. 14D compares the cytotoxicity of FdU versus CPVAp4FdU (NG-FU).

FIG. 15 provides graphs showing the tumor growth inhibition in mice with gemcitabine-resistant follicular lymphoma RL7/G tumors following the oral gavage treatment with the dual drug nanoformulation of CPVAp4FdU and gemcitabine (FIG. 15A) or the oral ad libitum treatment with CPVAp4FdU (FIG. 15B). The data were statistically significant between control and treatment groups.

### DETAILED DESCRIPTION OF THE INVENTION

Cytotoxic nucleoside analogues belong to the important class of anticancer drugs, which are currently used as the first line of treatment of hematological malignancies and certain solid tumors (Galmarini et al. (2002) Lancet Oncol., 3:415-424). These drugs act as antimetabolites by interfering with nucleic acid synthesis and enzymes of the nucleotide metabolism. The clinical efficacy of these drugs depends on higher metabolic activity and drug activation in rapidly proliferating cancer cells compared to normal cells. Activation of therapeutic nucleoside analogues occurs through the de novo synthesis of 5′-mono-, di-, and triphosphate derivatives, which interfere with the cellular pool of natural nucleosides. Nucleoside analogues require participation of specialized nucleoside transporter proteins such as hENT1, hENT2, or hCNT1 in order to accumulate in the cells. The integral drug uptake depends on the proper balance of the nucleoside transporters and drug efflux proteins presented on cellular membrane. Therefore, the drug accumulation is substantially reduced when the expression of such nucleoside transporters is deficient or the activity of drug efflux transporter proteins is elevated (Galmarini et al. (2002) Br. J. Hamaetol., 117:860-868; Ward et al. (2000) J. Biol. Chem., 275:8375-8381; Crawford et al. (1990) J. Biol. Chem., 265:13730-13734; Crawford et al. (1990) J. Biol. Chem., 265:9732-9736). After entering cells, nucleoside analogues undergo phosphorylation into 5′-monophosphates with deoxycytidine kinase (dCK) or thymidine kinase (TK), a rate-limiting step in the intracellular activation of nucleosides, and are subsequently converted into active 5′-diphosphates and 5′-triphosphates by other nucleoside kinases (Galmarini et al. (2002) Br. J. Hamaetol., 117:860-868). The efficacy of nucleoside analogues may further be limited by additional factors such as metabolic deamination and intracellular dephosphorylation (Funato et al. (2000) Leuk. Res., 24:535-541; Dumontet et al. (1999) Br. J. Hamaetol., 106:78-85).

In order to increase tumor accumulation of nucleoside analogues, various prodrug and drug delivery approaches have been developed, for example, the application of lipophilic nucleoside derivatives with an enhanced cellular membrane affinity. Many prodrugs with degradable lipophilic masking groups demonstrate the enhanced cell membrane permeability (Meier et al. (2006) Antiviral Res., 71:282-292). Early phase clinical trials have shown some improvements in the treatment of hematological malignancies, but these prodrugs were not effective in the treatment of solid tumors (Galmarini et al. (2008) Curr. Med. Chem., 15:1072-1082). Moreover, lipophilic prodrugs have a reduced half-life in circulation due to the fast accumulation in liver. Various nanocarriers such as liposomes, biodegradable nanoparticles, polymeric micelles, and nanocapsules have been extensively for tumor delivery of chemotherapeutic drugs (Zamboni, W. C. (2005) Clin. Cancer Res., 11:8230-8234). Many of these nanocarriers demonstrated advanced features, but have also shown serious shortcomings limiting their clinical usefulness. For example, liposomal formulations were unable to achieve effective drug concentration inside tumors because many anticancer drugs (cytarabine, 5-FU, etc.) diffused rapidly through the liposome bilayer (Crosasso et al. (1997) J. Pharm. Sci., 86:832-839).

An alternative tumor treatment strategy involves using formulation and nanodelivery of activated drugs, nucleoside 5′-triphosphates, encapsulated in cationic nanogels (Vinogradov et al. (2005) J. Controlled Release 107:143-157). In vivo delivery of bioactive 5′-triphosphates of nucleoside analogue have been attempted through encapsulation in liposomes, nanoparticles, or red blood cells (Duzgunes et al. (2005) Methods Enzymol., 391:351-373; Oussoren et al. (1999) Int. J. Pharm., 180:261-270; Vinogradov et al. (2010) Antivir. Chem. Chemother., 21:1-14; Magnani et al. (1997) J. Leukocyte Biol., 62:133-137). Evidently, the success of this strategy depends on the advantages of nanoformulations in drug protection in biological milieu, controlled drug release, and the specific tumor targeting. Nanogel carriers have dramatically improved the delivery of activated phosphorylated nucleoside analogues into cancer cells and tumor growth inhibition effect (Vinogradov et al. (2005) Mol. Pharm., 2:449-461; Galmarini et al. (2008) Mol. Cancer. Ther., 7:3373-3380). However, the noncovalent nature of the encapsulation of anionic 5′-triphosphates in cationic nanogels was the reason for relatively fast drug release kinetics.

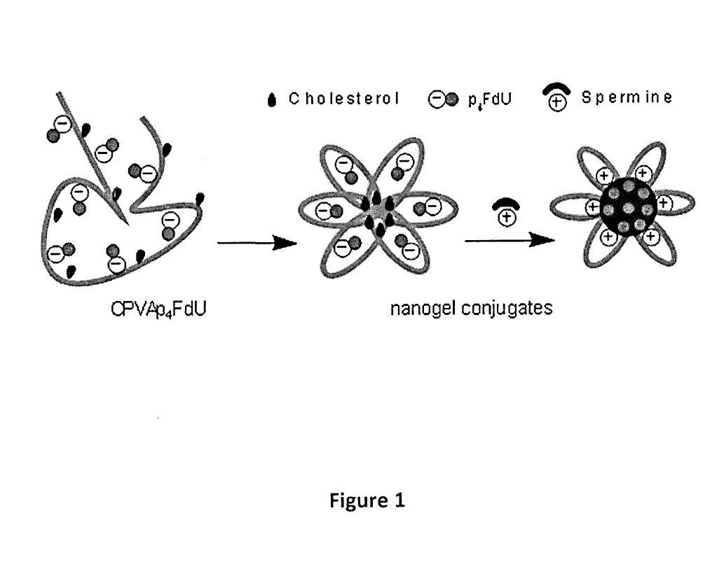

Here, the synthesis of novel types of drug-loaded nanogels, containing a polymer network with covalently linked phosphorylated nucleoside analogues, are reported which are capable of sustained drug release and are structurally different from the previously studied polymeric nanogels. Covalent drug attachment is an important factor in the controlled drug release, because it allows using specific chemistries. Various hydrolytically or enzymatically sensitive linkers, such as peptides, carboxylates, and so forth, have been evaluated with polymeric drug delivery systems (Aryal et al. (2010) Small 6:1442-1448; Ferey, G. (2008) Chem. Soc. Rev., 37:191-214; Ulbrich et al. (1982) Biomaterials 3:150-154). In this study, drug conjugates were synthesized by the attachment of nucleoside analogues through a biodegradable tetraphosphate linker starting from amphiphilic polymers such as cholesterol-modified polyvinyl alcohol (PVA) or dextrin (DEX). The linker has strong advantages over other linkers, because the polymeric drug conjugates are able to release nucleoside analogues in active phosphorylated form in the result of its hydrolytic or enzymatic degradation, eventually showing an enhanced tumor growth inhibition efficacy against normal and drug-resistant cancer cells. These drug-containing polymer conjugates can form stable nanogels with a small hydrodynamic diameter after ultrasonication in aqueous media (see FIG. 1). Selection of biodegradable or mucoadhesive biocompatible polymers for preparation of polymeric conjugates will also reduce toxicity of chemotherapy and allow for oral administration of these nanoformulations.

As stated hereinabove, inherent or therapy-induced drug resistance is a major clinical setback in cancer treatment. The extensive usage of cytotoxic nucleobases and nucleoside analogues in chemotherapy also results in the development of specific mechanisms of drug resistance, such as nucleoside transport or activation deficiencies. These drugs are prodrugs and are converted into the active mono-, di-, and triphosphates inside cancer cells following administration. They affect nucleic acid synthesis, nucleotide metabolism, or sensitivity to apoptosis. Nanodelivery of active nucleotide species, e.g., 5′-triphosphates of nucleoside analogues, can enhance drug efficacy and reduce nonspecific toxicity. Herein, a novel type of drug nanoformulations, polymeric conjugates of nucleoside analogues, is provided which are capable of the efficient transport and sustained release of phosphorylated drugs. These drug conjugates have been synthesized, starting from cholesterol-modified mucoadhesive polyvinyl alcohol or biodegradable dextrin, by covalent attachment of nucleoside analogues through a tetraphosphate linker. Association of cholesterol moieties in aqueous media resulted in intramolecular polymer folding and the formation of small nanogel particles containing 0.5 mmol/g (10-12% by nucleoside analog) of a 5′-phosphorylated nucleoside analogue (e.g., 5-fluoro-2′-deoxyuridine (floxuridine, FdU)) an active metabolite of anticancer drug 5-fluorouracyl (5-FU). The polymeric conjugates demonstrated rapid enzymatic release of floxuridine 5′-phosphate and much slower drug release under hydrolytic conditions (pH 1.0-7.4). Among the panel of cancer cell lines, all studied polymeric FdU-conjugates demonstrated an up to 125× increased cytotoxicity in human prostate cancer (PC-3), breast cancer (MCF-7, BT-474, and MDA-MB-231), and pancreatic cancer (MiaPaCa) cells, and more than 100× higher efficacy against cytarabine-resistant human T-lymphoma (CEM/araC/8) and gemcitabine-resistant follicular lymphoma (RL7/G) cells as compared to free drugs. In the initial in vivo screening, both PC-3 and RL7/G subcutaneous tumor xenograft models showed enhanced sensitivity to sustained drug release from polymeric FdU-conjugate after peritumoral injections and significant tumor growth inhibition. All these data demonstrate the clinical utility of the novel polymeric conjugates of phosphorylated nucleoside analogues, especially as new therapeutic agents against drug-resistant tumors.

In accordance with the instant invention, polymer conjugates (nanogels) are provided. In a particular embodiment, the polymer conjugate comprises a polymer conjugated to hydrophobic moieties and conjugated to at least one therapeutic agent via a phosphate linkage. In a particular embodiment, the therapeutic agent is a prodrug that is activated by conversion to a mono-, di- or triphosphate, particularly a di- or triphosphate. In a particular embodiment, the therapeutic agent is a nucleoside analog or nucleobase, particularly a cytotoxic nucleoside analog or nucleobase. The nucleoside analog may be an analog of a pyrimidine (e.g., cytosine, uracil, or thymine) or a purine (e.g., adenine or guanine). Examples of nucleoside analogs or nucleobases include, without limitation, floxuridine (5-fluoro-2′-deoxyuridine (FdU)), 5-fluorouracil, azidothymidine (AZT), cytarabine (cytosine arabinoside), gemcitabine, didanosine (2′,3′-dideoxyinosine, ddI), zalcitabine (dideoxycytidine; 2′,3′-dideoxycytidine, ddC), stavudine (2′,3′-didehydro-2′,3′-dideoxythymidine, d4T), lamivudine (2′,3′-dideoxy-3′-thiacytidine, 3TC), abacavir, apricitabine, emtricitabine (FTC), entecavir, arabinosyl adenosine (Ara-A), fluorouracil arabinoside, mercaptopurine riboside, 5-aza-2′-deoxycytidine, arabinosyl 5-azacytosine, 6-azauridine, azaribine, 6-azacytidine, trifluoro-methyl-2′-deoxyuridine, thymidine, thioguanosine, 3-deazautidine, 2-Chloro-2′-deoxyadenosine (2-CdA), 5-bromodeoxyuridine 5′-methylphosphonate, fludarabine (2-F-ara-AMP), 6-mercaptopurine, 6-thioguanine, 2-chlorodeoxyadenosine (CdA), 4′-thio-beta-D-arabinofuranosylcytosine, and salts and analogs thereof. In a particular embodiment, the nucleoside analog or nucleobase is selected from the group consisting of floxuridine, azidothymidine, 5-fluorouracil, abacavir, fludarabine, and gemcitabine.

In a particular embodiment of the instant invention, the polymer of polymer conjugates is a water-soluble polymer (hydrophilic). The polymer may be biodegradable and/or mucoadhesive. In a particular embodiment, the polymer comprises hydroxyl groups. For example, each repeating unit of the polymer may have one or more hydroxyl groups. The polymers of the instant invention may comprise a single repeating unit or may be copolymers such as block, random, alternating, or statistical copolymers. Examples of polymers of the instant invention include, without limitation, polyvinyl alcohol (PVA), polysaccharide, dextran, dextrin, cyclodextrin, polyethylene glycol (PEG) (including linear and branched PEGs including star-PEGs), poloxamers, Pluronic® (block copolymers of ethylene oxide and propylene oxide), pectin, chitin, chitosan, hyaluronic acid, and copolymers thereof.

The polymer of the instant invention is conjugated to at least one hydrophobic moiety. The hydrophobization of the polymer allows for the formation of micelles (e.g., “flower-type” micelles) or nanoparticles with internally aggregated hydrophobic moieties. In a particular embodiment, the degree of grafting of the hydrophobic moiety is at least about 0.1%, at least about 0.5%, at least about 1%, at least about 2%, at least about 5% or more. In a particular embodiment, the hydrophobized polymer comprises about 0.1% to about 25% or more, about 0.5% to about 15%, about 1% to about 10%, about 2% to about 8%, or about 4% to about 6% hydrophobic moiety by weight. The hydrophobic moiety can be coupled to the polymer by any means including, for example, linking with functional groups (e.g., hydroxyl groups) of the polymer. The hydrophobic moiety may be linked directly to the polymer or via a linker. Generally, the linker is a chemical moiety comprising a covalent bond or a chain of atoms that covalently attaches the ligand to the surfactant. The linker can be linked to any synthetically feasible position of the hydrophobic moiety and polymer. The linker may be non-degradable or degradable. The linker may be a covalent bond. The linker may be a chemical structure (e.g., esters or disulfide bonds) which can be substantially cleaved under physiological environments or conditions. Exemplary linkers may comprise at least one optionally substituted; saturated or unsaturated; linear, branched or cyclic alkyl group or an optionally substituted aryl group. In a particular embodiment, the hydrophobic moiety is a compound with a relatively low molecular weight (e.g., less than 4,000, less than 2,000, or less than 1 kDa or 800 Da). In a particular embodiment, the hydrophobic moiety is a lipid (e.g., phospholipid), fatty acid (e.g., docosahexaenoic acid (DHA)), retinoic acid, steroid, or cholesterol (e.g., cholesterol chloroformate). In a particular embodiment, the hydrophobic moiety comprises at least one linear, branched or cyclic alkyl group, alkenyl group, and/or at least one aryl group.

The formed nanoparticles of the instant invention may have a diameter of about 10 nm to about 1000 nm, particularly about 25 nm to about 500 nm, wherein the larger size particles are encapsulating a therapeutic agent (see below). To promote the formation of even smaller spherical nanoparticles, positively charged compounds (e.g., metal ions or salts or spermine) are added to the nanogel.

As stated hereinabove, the therapeutic agent is linked to the polymer via a phosphate linkage. The phosphate linkage may be a biodegradable polyphosphate linkage. In a particular embodiment, the phosphate linkage is a tetraphosphate linkage.

The conjugated polymers may also be conjugated to at least one targeting ligand, particularly on the outer portion of the formed micelle/nanoparticle. A targeting ligand is a compound that will specifically bind to a specific type of tissue or cell type. In a particular embodiment, the targeting ligand is a ligand for a cell surface marker/receptor. The targeting ligand may be an antibody or fragment thereof immunologically specific for a cell surface marker (e.g., protein or carbohydrate) preferentially or exclusively expressed on the targeted tissue or cell type (e.g., cancer cell). The targeting ligand can be coupled to the micelles by any means including, for example, linking with functional groups of the polymer. The targeting ligand may be linked directly to the micelle or via a linker. Generally, the linker is a chemical moiety comprising a covalent bond or a chain of atoms that covalently attaches the ligand to the polymer. The linker can be linked to any synthetically feasible position of the ligand and the non-ionic polymeric shell segments. Exemplary linkers may comprise at least one optionally substituted; saturated or unsaturated; linear, branched or cyclic alkyl group or an optionally substituted aryl group. The linker may also be a polypeptide (e.g., from about 1 to about 10 amino acids, particularly about 1 to about 5). The linker may be non-degradable (e.g., a covalent bond or any other chemical structure which cannot be substantially cleaved or cleaved at all under physiological environments or conditions) or degradable (e.g., a chemical structure which can be substantially cleaved under physiological environments or conditions). In a particular embodiment, the conjugated polymers comprise up to about 5%, up to about 10%, or up to about 15% of targeting ligands. Targeting ligands include, without limitation, receptor-specific peptides, folate, transferrin, streptavidine, insulin, aptamers (including modified aptamers such as thio-, 2′-fluoro, and 5-alkyl amino aptamers), and antibodies.

The nanogels of the instant invention may further comprise at least one other therapeutic agent. In a particular embodiment, the nanogel encapsulates the therapeutic agent. In a particular embodiment, the therapeutic agent is hydrophobic. The therapeutic agent may be a chemotherapeutic agent. Chemotherapeutic agents are compounds that exhibit anticancer activity and/or are detrimental to a cell (e.g., a toxin). Suitable chemotherapeutic agents include, but are not limited to: toxins (e.g., saporin, ricin, abrin, ethidium bromide, diptheria toxin, Pseudomonas exotoxin); alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, vinblastine, and paclitaxel (Taxol®)); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); HSP90 inhibitors (e.g., 17-AAG); and antihormonal antigens (e.g., tamoxifen, antiandrogen agents such as flutamide; and antiadrenal agents such as mitotane and aminoglutethimide). In a particular embodiment, the chemotherapeutic agent is paclitaxel, 17-AAG, or gemcitabine. In a particular embodiment, the therapeutic agent works synergistically with the therapeutic agent attached to the polymer and/or a second therapeutic agent encapsulated by the nanogel.

Methods of synthesizing the nanogels of the instant invention are encompassed herein. In a particular embodiment, the method comprises at least partially hydrophobizing a hydrophilic polymer, conjugating at least one phosphate linker to the polymer, and conjugating at least one therapeutic agent to the phosphate linker. The methods may further comprise contacting the polymer with a counter ion (e.g., a cation such as spermine, polyamino acids (e.g., poly amino acids of positive amino acids (e.g., polylysine), epsilon-polylysine, and poly(ethyleneimine) (PEI) and derivates thereof (e.g., disulfide bond containing PEI). The methods may further comprise sonicating (e.g., in an aqueous solution) the resultant nanogel to promote nanoparticle formation. In a particular embodiment, the phosphate linker is added to the polymer via a stepwise method from polymer-phosphate, to polymer-triphosphate, and then to polymer-tetraphosphate-therapeutic agent. Phosphorylation steps may be performed using phosphorylating reagents such as phosphoryl chloride (POCl₃), phosphoryl tris-imidazolyde, and 2-cyanoethyl phosphoryl bis-imidazolyde. In a particular embodiment, the methods further comprise conjugating at least one targeting ligand to the polymer. In a particular embodiment, the methods further comprise incorporating at least one other therapeutic agent into the nanogel (e.g., into the core of the nanogel/nanoparticle, such as forming nanogel/nanoparticles in the presence of the therapeutic agent).

The instant invention encompasses compositions comprising at least one nanogel (nanoparticle) of the instant invention and at least one pharmaceutically acceptable carrier. As stated hereinabove, the nanogel may comprise more than one therapeutic agent. The compositions of the instant invention may further comprise other therapeutic agents (e.g., other chemotherapeutic agents).

The present invention also encompasses methods for preventing, inhibiting, and/or treating a disease or disorder, particularly cancer. The methods comprise administering at least one nanogel of the instant invention (e.g., in a composition with a pharmaceutically acceptable carrier) to a subject in need thereof. In a particular embodiment, the cancer is prostate, pancreatic, breast, hematological, or colon cancer. In a particular embodiment, the cancer is resistant to one or more drugs or therapeutic agents, particularly at least one cytotoxic nucleoside analog (e.g., the same cytotoxic nucleoside analog being administered as part of the nanogel of the instant invention).

The pharmaceutical compositions of the instant invention are administered to an animal, in particular a mammal, more particularly a human, in order to treat/inhibit/prevent the disease or disorder. The pharmaceutical compositions of the instant invention may also comprise at least one other therapeutic agent. The additional agent may also be administered in separate composition from the nanogels of the instant invention. The compositions may be administered at the same time or at different times (e.g., sequentially). When the disease is cancer, the compositions of the instant invention may also be administered with chemoradiation (e.g., sequentially).

In a particular embodiment, the disease or disorder is a viral infection. In a particular embodiment, the virus is a retrovirus, particularly a lentivirus, particularly HIV. The nanogels of the instant invention, particularly when used to treat/inhibit/prevent a viral infection, may encapsulate an anti-viral compound, particularly an antiretroviral or anti-HIV compound. As used herein, an “anti-HIV compound” is a compound which inhibits HIV. Examples of an anti-HIV compound include, without limitation: nucleoside-analog reverse transcriptase inhibitors, non-nucleoside reverse transcriptase inhibitors (NNRTIs), protease inhibitors, and fusion inhibitors. As used herein, the term “nucleoside-analog reverse transcriptase inhibitors” (NRTIs) refers to nucleosides and nucleotides and analogues thereof that inhibit the activity of HIV-1 reverse transcriptase. As used herein, NNRTIs are allosteric inhibitors which bind reversibly at a nonsubstrate-binding site on the HIV reverse transcriptase, thereby altering the shape of the active site or blocking polymerase activity. As used herein, the term “protease inhibitor” refers to inhibitors of the HIV-1 protease. As used herein, “fusion inhibitors” are compounds, such as peptides, which act by binding to HIV envelope protein and blocking the structural changes necessary for the virus to fuse with the host cell.

In accordance with another aspect of the instant invention, ATP-conjugated polymers of the instant invention (e.g., adenosine attached to the polyphosphate linker (e.g., tetraphosphate)) may be used as cellular energy sources in the treatment of a neurodegenerative disease or disorder. Examples of neurodegenerative diseases or disorders include, without limitation, stroke, Alzheimer's, Parkinson's, meningitis, and HIV (e.g., HIV-related neurodegenerative disease (HRND)).

The dosage ranges for the administration of the compositions of the invention are those large enough to produce the desired effect. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications.

The nanogels described herein will generally be administered to a patient as a pharmaceutical preparation. The term “patient” as used herein refers to human or animal subjects. These nanogels may be employed therapeutically, under the guidance of a physician.

The compositions comprising the nanogels of the instant invention may be conveniently formulated for administration with any pharmaceutically acceptable carrier(s). For example, the nanogels may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the nanogels in the chosen medium may be varied and the medium may be chosen based on the desired route of administration of the pharmaceutical preparation. Except insofar as any conventional media or agent is incompatible with the nanogels to be administered, its use in the pharmaceutical preparation is contemplated.

The dose and dosage regimen of nanogels according to the invention that are suitable for administration to a particular patient may be determined by a physician considering the patient's age, sex, weight, general medical condition, and the specific condition for which the nanogels are being administered and the severity thereof. The physician may also take into account the route of administration, the pharmaceutical carrier, and the biological activity of the nanogels.

Selection of a suitable pharmaceutical preparation will also depend upon the mode of administration chosen. For example, the nanogels of the invention may be administered by direct injection (e.g., intratumor or to the surrounding area), orally, or intravenously. In the instance of direct injection, a pharmaceutical preparation comprises the nanogels dispersed in a medium that is compatible with the site of injection.

Nanogels of the instant invention may be administered by any method. For example, the nanogels of the instant invention can be administered, without limitation parenterally, subcutaneously, orally, topically, pulmonarily, rectally, vaginally, intravenously, intraperitoneally, intrathecally, intracerbrally, epidurally, intramuscularly, intradermally, or intracarotidly. In a particular embodiment, the nanogels are administered intravenously, orally, or intraperitoneally. Pharmaceutical preparations for injection are known in the art. If injection is selected as a method for administering the nanogels, steps must be taken to ensure that sufficient amounts of the molecules reach their target cells to exert a biological effect. Dosage forms for oral administration include, without limitation, tablets (e.g., coated and uncoated, chewable), gelatin capsules (e.g., soft or hard), lozenges, troches, solutions, emulsions, suspensions, syrups, elixirs, powders/granules (e.g., reconstitutable or dispersible) gums, and effervescent tablets. Dosage forms for parenteral administration include, without limitation, solutions, emulsions, suspensions, dispersions and powders/granules for reconstitution. Dosage forms for topical administration include, without limitation, creams, gels, ointments, salves, patches and transdermal delivery systems.

Pharmaceutical compositions containing nanogels of the present invention as the active ingredient in intimate admixture with a pharmaceutically acceptable carrier can be prepared according to conventional pharmaceutical compounding techniques. The carrier may take a wide variety of forms depending on the form of preparation desired for administration, e.g., intravenous, oral, direct injection, intracranial, and intravitreal.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of nanogels may be determined by evaluating the toxicity of the molecules or cells in animal models. Various concentrations of nanogels in pharmaceutical preparations may be administered to mice, and the minimal and maximal dosages may be determined based on the beneficial results and side effects observed as a result of the treatment. Appropriate dosage unit may also be determined by assessing the efficacy of the nanogel treatment in combination with other standard drugs. The dosage units of nanogels may be determined individually or in combination with each treatment according to the effect detected.

The pharmaceutical preparation comprising the nanogels may be administered at appropriate intervals, for example, at least twice a day or more until the pathological symptoms are reduced or alleviated, after which the dosage may be reduced to a maintenance level. The appropriate interval in a particular case would normally depend on the condition of the patient.

The instant invention encompasses methods of treating a disease/disorder comprising administering to a subject in need thereof a composition comprising nanogels of the instant invention and, particularly, at least one pharmaceutically acceptable carrier. Other methods of treating the disease or disorder may be combined with the methods of the instant invention may be co-administered with the compositions of the instant invention. The instant invention also encompasses ex vivo methods of treatment. The instant invention also encompasses delivering the nanogels of the instant invention to a cell in vitro (e.g., in culture). The nanogels may be delivered to the cell in at least one carrier.

### DEFINITIONS

As used herein, the term “nanogel” refers to a hydrophobized polymer gel nanoparticle comprising a hydrophilic polymer with hydrophobic moieties added thereto, particularly as a side chain.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions may be employed as carriers, particularly for injectable solutions. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition (e.g., cancer) resulting in a decrease in the probability that the subject will develop the condition.

In one embodiment “drug-resistant cancer” refers to cancer cells that acquire resistance to chemotherapy. Cancer cells can acquire resistance to chemotherapy by a range of mechanisms including, without limitation, a deficiency in membrane nucleoside transporters or the overexpression or over-activity of drug efflux pumps (i.e., elimination of the drug from the cell), inactivation of the drug, and induction of anti-apoptotic mechanisms.

As used herein, the term “subject” refers to an animal, particularly a mammal, particularly a human.

A “therapeutically effective amount” of a compound or a pharmaceutical composition refers to an amount effective to prevent, inhibit, treat, or lessen the symptoms of a particular disorder or disease. The treatment of cancer herein may refer to curing, relieving, and/or preventing the cancer, the symptom(s) of it, or the predisposition towards it.

As used herein, the term “therapeutic agent” refers to a chemical compound or biological molecule including, without limitation, nucleic acids, peptides, proteins, and antibodies that can be used to treat a condition, disease, or disorder or reduce the symptoms of the condition, disease, or disorder.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, or nucleic acids, though they may be amino acids or dipeptides.

As used herein, the term “amphiphilic” means the ability to dissolve in both water and lipids/apolar environments. Typically, an amphiphilic compound comprises a hydrophilic portion and a hydrophobic portion. “Hydrophobic” designates a preference for apolar environments (e.g., a hydrophobic substance or moiety is more readily dissolved in or wetted by non-polar solvents, such as hydrocarbons, than by water). As used herein, the term “hydrophilic” means the ability to dissolve in water.

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

An “antibody” or “antibody molecule” is any immunoglobulin, including antibodies and fragments thereof (e.g., scFv), that binds to a specific antigen. As used herein, antibody or antibody molecule contemplates intact immunoglobulin molecules, immunologically active portions of an immunoglobulin molecule, and fusions of immunologically active portions of an immunoglobulin molecule.

As used herein, the term “immunologically specific” refers to proteins/polypeptides, particularly antibodies, that bind to one or more epitopes of a protein or compound of interest, but which do not substantially recognize and bind other molecules in a sample containing a mixed population of antigenic biological molecules.

The term “alkyl,” as employed herein, includes straight, branched, and cyclic chain hydrocarbons containing 1 to about 20 carbons or 1 to about 10 carbons in the normal chain. The hydrocarbon chain of the alkyl groups may be interrupted with one or more oxygen, nitrogen, or sulfur. Examples of suitable alkyl groups include methyl, ethyl, propyl, isopropyl, butyl, t-butyl, isobutyl, pentyl, hexyl, isohexyl, heptyl, 4,4 dimethylpentyl, octyl, 2,2,4 trimethylpentyl, nonyl, decyl, the various branched chain isomers thereof, and the like. Each alkyl group may, optionally, be substituted, preferably with 1 to 4 substituents. The term “lower alkyl” refers to an alkyl which contains 1 to 3 carbons in the hydrocarbon chain. The term “cyclic alkyl” or “cycloalkyl,” as employed herein, includes cyclic hydrocarbon groups containing 1 to 3 rings which may be fused or unfused. Cycloalkyl groups may contain a total of 3 to 20 carbons forming the ring(s), particularly 6 to 10 carbons forming the ring(s). Optionally, one of the rings may be an aromatic ring as described below for aryl. The cycloalkyl groups may also, optionally, contain substituted rings that includes at least one (e.g., from 1 to about 4) sulfur, oxygen, or nitrogen heteroatom ring members. Each cycloalkyl group may be, optionally, substituted, with 1 to about 4 substituents. Alkyl substituents include, without limitation, alkyl, alkenyl, halo (such as F, Cl, Br, I), haloalkyl (e.g., CCl₃ or CF₃), alkoxyl, alkylthio, hydroxy, methoxy, carboxyl, oxo, epoxy, alkyloxycarbonyl, alkylcarbonyloxy, amino, carbamoyl (e.g., NH₂C(═O)— or NHRC(═O)—, wherein R is an alkyl), urea (—NHCONH₂), alkylurea, aryl, ether, ester, thioester, nitrile, nitro, amide, carbonyl, carboxylate and thiol. In a particular embodiment, the substituent is hydrophobic such as an alkyl or aryl.

“Alkenyl” refers to an unsubstituted or substituted hydrocarbon moiety comprising one or more carbon to carbon double bonds (i.e., the alkenyl group is unsaturated) and containing from 1 to about 20 carbon atoms or from 1 to about 10 carbon atoms, which may be a straight, branched, or cyclic hydrocarbon group. The hydrocarbon chain of the alkenyl groups may be interrupted with one or more oxygen, nitrogen, or sulfur. When substituted, alkenyl groups may be substituted at any available point of attachment. Exemplary substituents are described above for alkyl groups.

The term “aryl,” as employed herein, refers to monocyclic and bicyclic aromatic groups containing 6 to 10 carbons in the ring portion. Examples of aryl groups include, without limitation, phenyl, naphthyl, such as 1-naphthyl and 2-naphthyl, indolyl, and pyridyl, such as 3-pyridyl and 4-pyridyl. Aryl groups may be optionally substituted through available carbon atoms, preferably with 1 to about 4 groups. Exemplary substituents are described above for alkyl groups. The aryl groups may be interrupted with one or more oxygen, nitrogen, or sulfur heteroatom ring members (e.g., a heteroaryl).

The following examples provide illustrative methods of practicing the instant invention, and are not intended to limit the scope of the invention in any way.

### Example 1

### Materials and Methods

### Materials

Most reagents, solvents, and polymers were purchased from Sigma Aldrich (St. Louis, Mo.) and Alfa Aesar (Wardhill, Mass.) with the highest available purity and used without purification unless otherwise stated. Thymidine, 3-[4,5-dimethylthiazol-2-yl]-2,5-diphenyltetrazolium bromide (MTT) and snake venom phosphodiesterase 1, type VI, from Carotalus adamanteus were purchased from Sigma (St. Louis, Mo.). Nucleoside analogues: 5-fluoro-2′-deoxyuridine (Floxuridine, FdU) was from SynQuest Laboratories (Alachua, Fla.), 2,2′-difluorocytidine (dFdC, Gemcitabine) was from Beta Pharma, Inc. (Branford, Conn.), and arabinosylcytosine (araC, Cytarabine) was from 3B Medical Systems, Inc. (Libertyville, Ill.). Centrifuge filter devices (MWCO 5000 Da) were purchased from Millipore (Bedford, Mass.).

All NMR spectra were recorded using a 500 MHz Varian NMR spectrometer. All chemical shift values are given in parts per million (ppm) and are referenced to a signal from (CH₃)₄Si (0 ppm) for ¹H, DMSO-d₆ (39.7 ppm) for ¹³C, and 85% phosphoric acid (0 ppm) for ³¹P spectra at 25° C. Hydrodynamic diameter, polydispersity, and zeta potential of nanogels and polymeric conjugates were measured using a dynamic light scattering instrument, the Zetasizer Nano-ZS90 (Malvern Instruments, Southborough, Mass.) at 25° C. Monodisperse polystyrene dispersions were used as standards. UV absorbance of samples was measured by Biophotometer (Eppendorf, Hamburg, Germany). IR spectra were recorded using a Nicolet IR-200 FTIR spectrometer (Thermo Scientific, Waltham, Mass.).

### Cells

Human breast carcinoma MCF-7, human hepatocellular carcinoma HepG2, and human prostate adenocarcinoma PC-3 cells were obtained from ATCC (Rockville, Md.). These cells were maintained in Dulbecco's Modified Eagle Medium (DMEM) supplemented with 10% fetal bovine serum (FBS), 1% L-glutamine, and 2% penicillin-streptomycin at 37° C. in a humidified atmosphere containing 5% CO₂. Human breast carcinoma MDA-MB-231 cell line was a gift from Dr. R. Singh (UNMC). These cells were maintained in DMEM/Nutrient mixture F-12 (DMEM/F12) with similar supplements and serum as above. Gemcitabine-resistant human follicular lymphoma RL7/G cell line, which is characterized by a reduced level of dCK enzyme (Galmarini et al. (2004) BMC Pharmacol., 4:8), was a gift from Dr. F. Bontemps (De Duve Institute, Bruxelles, Belgium). They were grown in the presence of 2 μM gemcitabine. Nucleoside transport-deficient cytarabine-resistant human leukemic lymphoblast CEM/araC/8 cell line (Ullman, B. (1989) Adv. Exp. Med. Biol., 253B:415-420) was obtained from Dr. C. Galmarini (UFR Lyon-Sud, Oullins, France). The cells were grown in the presence of 0.5 μM cytarabine (araC). Both drug-resistant cell lines were grown in RPMI medium supplemented with 10% fetal bovine serum (FBS), 1% L-glutamine, and 2% penicillin-streptomycin at 37° C. in a humidified atmosphere containing 5% CO₂.

### Synthesis of Cholesterol Conjugates

PVA was grafted with cholesterol moieties according to the procedures described below. Briefly, 2.1 g of PVA (M_{w} 13 kDa) was dried over phosphorus pentoxide in vacuo and dissolved in 50 mL of anhydrous DMSO at 70° C. Triethylamine (0.8 mmol) was added to the cooled solution (25° C.) followed by 0.3 g (0.68 mmol) of cholesteryl chloroformate, and the final solution was stirred overnight at 25° C. The reaction mixture was concentrated in vacuo and dialyzed (MWCO 3.5 kDa) against 20% aqueous ethanol three times for 24 hours. The product (CPVA) was isolated after concentration in vacuo and freeze-drying with a yield of 80%. In another method, 2.1 g of PVA (Mw 13 kDa) was dissolved in 50 mL of anhydrous N-methylpyrrolidone at 70° C., then 0.3 g (0.68 mmol) of cholesteryl chloroformate was added, and the mixture was stirred for 4 hours at 70° C. The substituted CPVA was precipitated in diethyl ether (0.5 L) and dried in vacuo; the light yellow precipitate was obtained at a yield of 70%. ¹H NMR: 0.63 (s, 18H), 0.83 (m, 36H), 0.88 (m, 18H), 0.92 (s, 18H), 1.11-1.95 (m, 764H), 3.84-3.90 (m, 295H), 4.31 (brs, OH), 4.40 (s, 12H), 5.25 (s, 6H). Following the same protocol for PVA (M_{w} 31 kDa), CPVA was obtained with a yield of 85%. ¹H NMR: 0.65 (s, 18H), 0.83-0.85 (dd, J=5.0, 1.6 Hz, 36H), 0.89 (d, J=4.8 Hz, 18H), 0.94 (s, 18H), 1.07-1.98 (m, 1582H), 3.84 (m, 704H), 4.35 (12H and OH), 5.28 (s, 6H). IR: 3264, 2897, 1642, 1409, 1323, 1082, 915, 829.

The dextrin-cholesterol nanogel (CDex) was synthesized as follows. The water-soluble fraction of dextrin (M_{w} 9 kDa) was isolated by dialysis in a Spectra/Pore membrane tube (MWCO 2 kDa) followed by centrifugation. The supernatant was freeze-dried and used for nanogel synthesis. 1.0 g of the purified dextrin was dried over phosphorus pentoxide in vacuo and dissolved in 15 mL of anhydrous DMSO at 70° C. After 0.3 g (0.68 mmol) of cholesteryl chloroformate was added, the reaction mixture was stirred for 24 hours at 25° C., concentrated in vacuo, and dialyzed (MWCO 3.5 kDa) against 20% aqueous ethanol three times for 24 hours. The product (CDex) was isolated after concentration in vacuo and freeze-drying with a yield of 76%. ¹H NMR: 0.65 (s, 18H), 0.83-0.85 (dd, J=5.0, 1.6 Hz, 36H), 0.89-1.51 (m, 211H), 3.24-3.64 (m, 333H), 4.28-5.10 (m, 122H), 5.23 (s, 6H), 5.37-5.62 (m, 551-1).

### Synthesis of Phosphorylating Reagent, CNEtOP(O)Im₂

The intermediate product CNEtOP(O)Cl₂ was synthesized by dissolving 18.6 mL (30.6 g, 0.2 mol) phosphorus (V) oxychloride and 20.6 mL (14.84 g, 0.147 mol) triethylamine in 40 mL anhydrous tetrahydrofuran (THF) at 0° C. It was treated with 10 mL (10.45 g, 0.147 mol) 2-cyanoethanol in 5 mL THF while stirring at 0° C. Stirring was continued for 30 minutes until white precipitate formed. The precipitate was carefully filtered with exclusion of moisture, and the resulting solution was concentrated in vacuo and distilled under argon. The product, CNEtOP(O)Cl₂, was recovered by distillation at 90° C./1 mm with a yield of 60%.

The product, CNEtOP(O)Im₂, was synthesized by mixing 2.69 g (0.015 mol) CNEtOP(O)Cl₂ and 5.25 g (0.037 mol) N-trimethylsilyl-imidazole in 40 mL cold anhydrous toluene. The solutions were then incubated for 2 hours at room temperature, concentrated in vacuo to a half-volume and placed in a freezer for 2 hours at −20° C. The precipitate of CNEtOP(O)Im₂ was recovered after centrifugation with a yield of 70% (Sinha et al. (1984)

### Nucleic Acids Res., 12:4539-4557).

### Preparation of Polymeric Drug Conjugates

A solution of 3.3 g of dried cholesterol-polymer conjugates in 33 mL DMF was treated with a 2M solution of CNEtOP(O)Im₂ in anhydrous DMF (2 mL) for 30 minutes at 25° C. Then, a 1 M solution of tetran-butylammonium salt of pyrophosphate (PPi-TBA) in anhydrous DMF (4 mL) was added, and the reaction mixture was incubated for 1 hour at 25° C. In a separate flask, floxuridine (FdU, 490 mg, 2 mmol) was treated with a 2 M solution of CNEtOP—(O)Im₂ in anhydrous DMF (1 mL) and allowed to stand for 20 minutes at 25° C. Both solutions were then mixed and stirred for 40 minutes at 25° C. The reaction mixture was treated with 1 mL of methanol and left overnight at 4° C. Insoluble material was removed by filtration; the nanogel conjugate was purified three times over 24 hours by dialysis (MWCO 3500 Da) against 20% aqueous ethanol, concentrated in vacuo, and precipitated as a sodium perchlorate in acetone. FdU content in the nanogel conjugate was measured by UV absorbance (ε₂₆₀=7570). Drug loading: CPVA31, 0.51 μmol/mg; CPVA13, 0.50 μmol/mg; and CDex9, 0.44 μmol/mg.

### Particle Size and Zeta-Potential Measurements

The hydrodynamic diameter and polydispersity of nanogels and polymeric conjugates were measured by dynamic light scattering (DLS) using a Zetasizer Nano-ZS90 with a 15 mV solid state laser operated at a wavelength of 635 nm. In brief, dry samples were resuspended in filtered deionized water, and then sonicated for 1 hour at 4° C. to form a uniform dispersion of nanoparticles and centrifuged for 4 minutes at 10,000×g. The size distribution in samples was characterized by polydispersity index. Zeta-potential was calculated based on electrophoretic mobility measurements performed with an electrical field strength of 15-18 V cm⁻¹ at 25° C. using the instrument software. The data reported in Table 1 represent an average of three measurements.

### Enzymatic Hydrolysis

Enzymatic stability and drug release from polymeric conjugates was assayed in 50 μL reaction mixtures containing: 100 mM Tris-HCl (pH 8.75), 2 mM MgCl₂, 0.5 mg of snake venom phosphodiesterase (VPDE), and 0.5 mg nanogel sample (FdU, 0.25 μmol). The reaction mixture was incubated at 37° C. and, at appropriate times, 5 μL aliquots were taken out and quenched with 1.5 μL of 1 M HCl. Nucleotide content was analyzed by ion-pair HPLC using an Ascentis C 18 column (10 μm, 15 cm×4.6 mm) at a flow rate of 1 mL/min. The elution was performed with buffer A: 40 mM KH₂PO₄, 0.2% tetrabutylammonium hydroxide, pH 7.0, and buffer B: 30% acetonitrile, 40 mM KH₂PO₄, 0.2% tetrabutylammonium hydroxide, pH 7.0, in a linear gradient mode (100% B in 20 minutes).

### In Vitro Drug Release

In vitro drug release was investigated under different pH values. In short, 18 mg of CPVA31-p₄FdU or CDex9-p₄FdU conjugates was dissolved in 20 mL of PBS solution at pH 7.4, 4.0, and 1.0. These solutions were incubated at 37° C. and 0.5 mL aliquots were taken out every 24 hours. The released nucleotides were separated from the rest of the polymeric conjugates by centrifugation at 7500 rpm for 25 minutes using an Amicon Ultra 0.5 centrifuge filter device (MWCO 3000 Da). The pH of the filtrate was adjusted to 7.4 and UV absorbance was measured at 260 nm.

### Cytotoxicity Studies

Cytotoxicity of the polymeric conjugates was analyzed in different cancer cell lines by a standard MTT assay. Briefly, MCF-7, PC-3, HepG2, and MDA-MB-231 cells were seeded at a density of 10 000 cells/200 μL growth medium/well in flat-bottom 96-well plates; the corresponding suspensions of RL7/G and CEM/araC/8 cells were placed in round-bottom 96-well plates. Cells were allowed to grow overnight and appropriate amounts of drug, nanogels, or polymeric conjugates were added. Samples were incubated in full medium for 72 hours at 37° C., and the metabolic activity of each sample was determined by adding 20 μL of a 5 mg/mL of MTT stock solution in sterile PBS buffer to each well. The samples were then incubated for 2 hours at 37° C., the medium and the MTT dye were washed out by PBS, and 1004 of extraction buffer (20% w/v SDS in DMF/water, 1:1, pH 4.7) was added to each well. Samples were incubated for 24 hours at 37° C. Optical absorbance was measured at 560 nm using a model 680 microplate reader (BioRad, Hercules, Calif.) and cytotoxicity was expressed as a percentage of survived cells relative to nontreated control cells. All samples were analyzed by an average of eight measurements (means±SEM). These data were plotted versus drug/nanogel concentrations and converted into IC₅₀ values (concentration of the 50% cell survival).

### In Vivo Tumor Growth Inhibition Assay

These experiments were performed using female nu/nu mice (RL7/G cells) or male nu/nu mice (PC-3 cells), aged 6-8 weeks (Charles River Laboratories, Wilmington, Mass.). Animal studies were carried out according to the Principles of Animal Care outlined by the National Institutes of Health, and protocols were approved by the Institutional Animal Care and Use Committee at the University of Nebraska Medical Center. The animals were randomly divided into groups of five per cage and maintained under sterile conditions and 12 hour light/dark cycle in a temperature-controlled environment. All manipulations with animals were performed in a sterile laminar hood using sterile solutions. PC-3 and RL7/G cell suspensions of 5×10⁶ cells/400 μL of medium containing 20% Matrigel (Becton-Dickinson, SanDiego, Calif.) were injected subcutaneously in the right flank areas of mice. After tumors could be palpitated, the treatment solutions of CPVA31-p₄FdU were injected peritumorally (2×100 μL) twice a week at a dose of 12 mg FdU/kg. Tumor volume was measured by digital calipers and calculated based on the equation: TV=L/2×W², where L and W are length and width of tumor (mm).

### Results

### Synthesis of Nanogel Conjugates

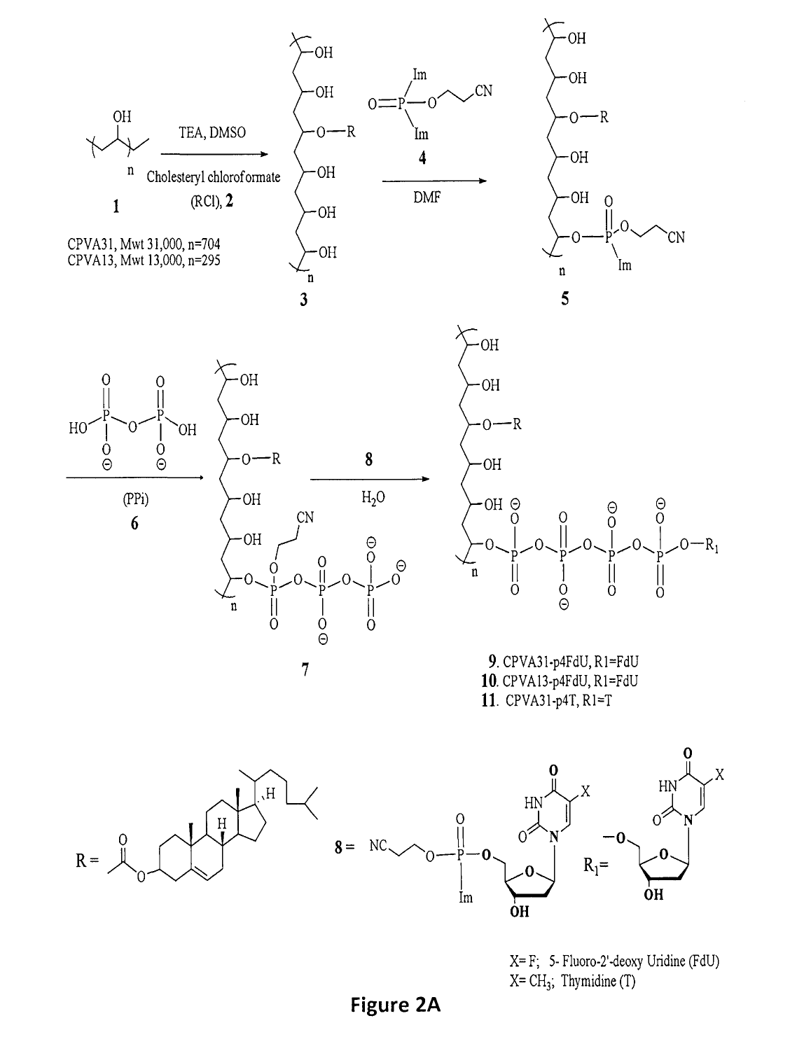

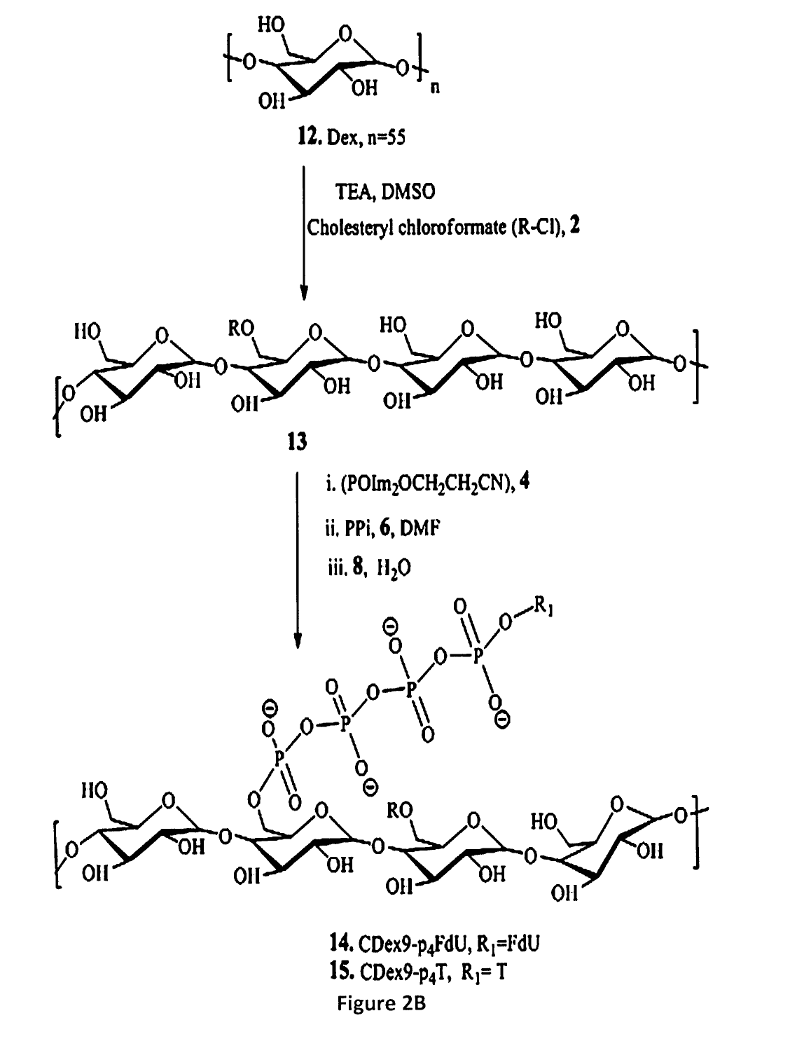

Cholesterol is a well-known hydrophobic moiety used in many drug delivery applications in order to enhance the interactions of modified macromolecules or nanocarriers with the cellular membrane (Vinogradov et al. (1994) Biochem. Biophys. Res. Commun., 203:959-966; Nochi et al. (2010) Nat. Mater., 9:572-578). In the instant design of polymeric conjugates, polymer modification with cholesterol has been exploited for several reasons: (i) to render hydrophilic polymers soluble in organic solvents, (ii) to compel the modified polymers to form compact nanogels, and (iii) to increase membranotropic properties and ease transport of hydrophilic drug molecules across the cellular membrane. In aqueous solutions, at ultrasonication cholesterol-modified polymers form “flower-type” micelles with internally aggregated cholesterolmoieties with the least association numbers 4-6 (Yusa et al. (1998) Langmuir 14:6059-6067). The cholesterol-modified polyvinyl alcohol (CPVA) containing six hydrophobic moieties per polymer chain were synthesized by reaction of the corresponding PVA, M_{w} 13 and 31 kDa, with cholesterol chloroformate in dry DMSO in the presence of triethylamine at room temperature. The cholesterol-modified PVA polymers were isolated with a yield of 80-85%. In an alternative method, PVA was modified in N-methylpyrrolidone at 70° C., yielding the corresponding cholesterol-modified polymer at 70-75% as a white solid after precipitation in diethyl ether (Gimenez et al. (1999) Polymer 40:2759-2767). The cholesterol-modified PVA polymers were designated as CPVA13 and CPVA31 (FIG. 2A). Similarly, as shown in FIG. 2B, a cholesterol-modified dextrin (CDex) was synthesized with a high yield starting from dextrin (Mw 9 kDa). Four cholesterol moieties were attached to the smaller dextrin molecule. The flexibility of charged polymer chains was restricted by hydrophobic cholesterol groups aggregated in the core of nanogels. The nanogels bearing negatively charged phosphate groups formed even smaller spherical compacted particles following the addition of positively charged spermine molecules (FIG. 1).

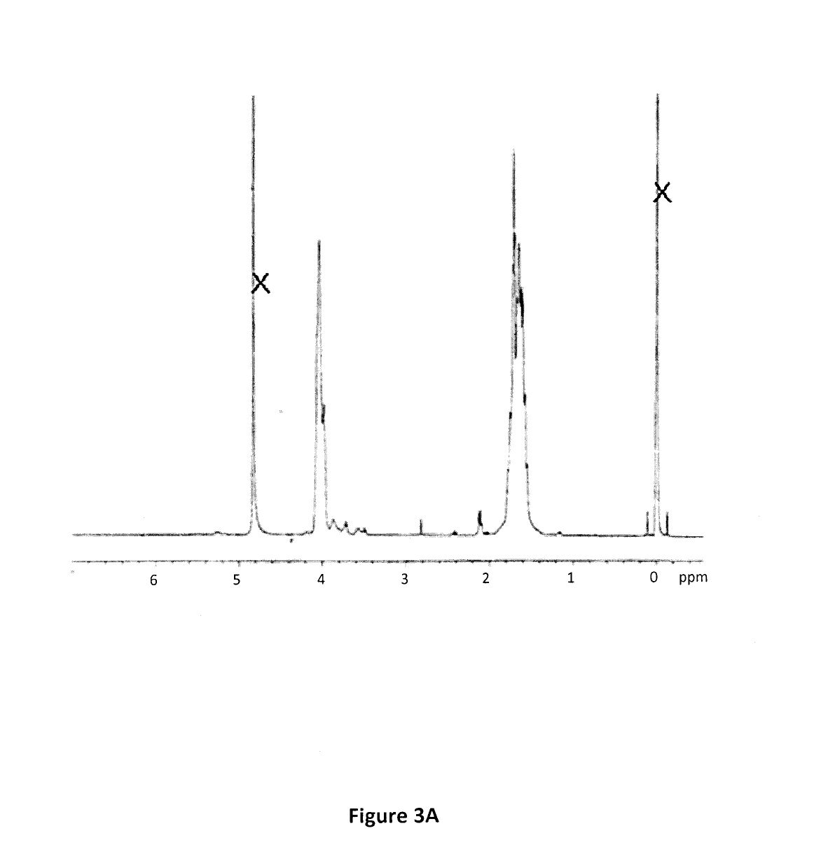

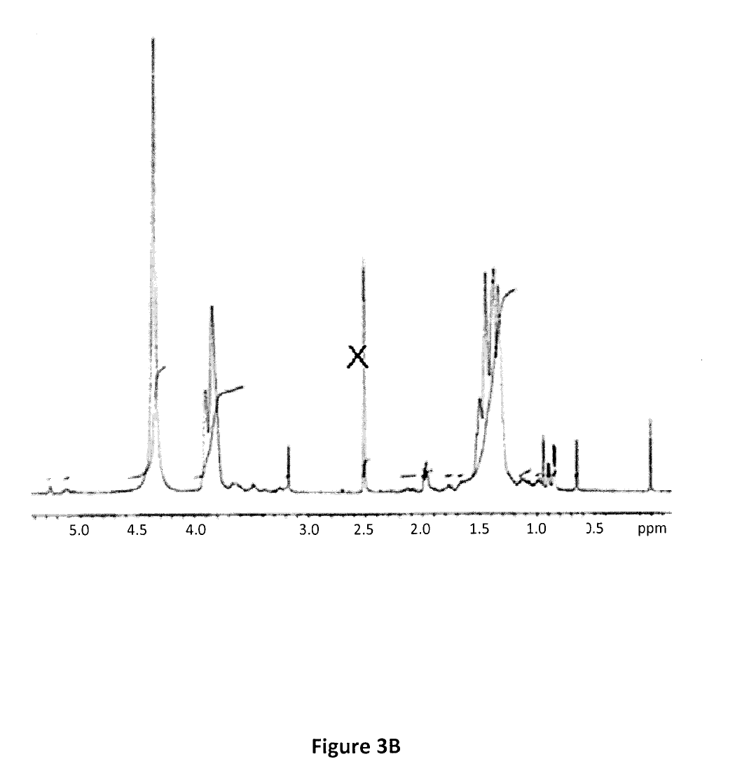

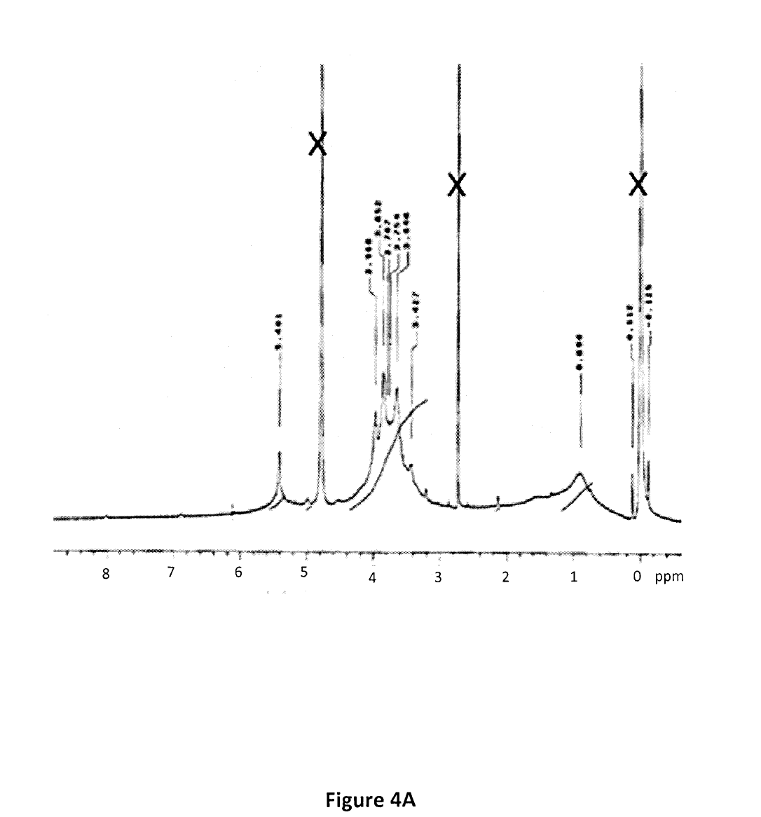

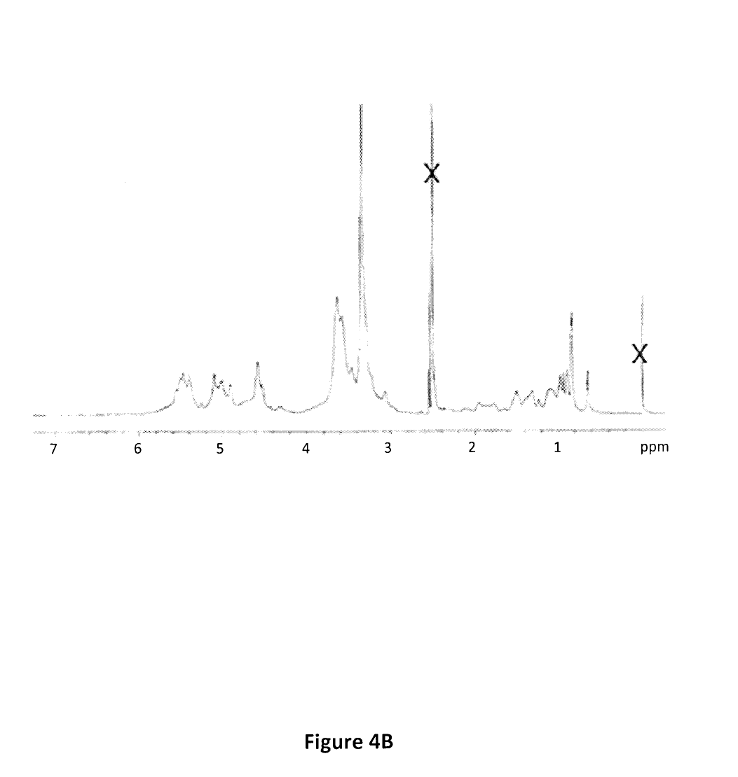

¹H NMR spectra showed that the cholesterol modification was nearly quantitative and amounted for six moieties per polymer chain in CPVA13 and CPVA31 and four per polymer chain in CDex9. Similarly, the formation of micelles in aqueous solutions by these polymers was also demonstrated by ¹H NMR spectroscopy. FIGS. 3 and 4 represent the spectra of CPVA13 and CDex9 in (a) D₂O and (b) DMSO-d₆, respectively. As shown in the spectra, proton signals of the cholesterol moiety (δ=0.6-2.4 ppm) appeared in DMSO-d₆ (b), but completely disappeared or wide broadening of signals was observed in D₂O (a). This indicates the restricted molecular motion of cholesterol moieties upon self-aggregation. The results confirmed the formation of a rigid core of hydrophobic cholesterol moieties and a relatively mobile shell consisting of hydrophilic PVA or Dex molecules in aqueous medium. The degree of cholesterol substitution (DS) was evaluated by calculating the ratio between the integrals of the protons in terminal CH₃-groups of cholesterol and methylene protons in CPVA or the protons of sugar monomers in CDex.

The hydroxyl functional groups in the cholesterol-modified polymers have been used as sites for the conjugation of the active phosphorylated nucleoside analogue, floxuridine (FdU), resulting in the formation of polymeric conjugates as anticancer drug carriers (FIGS. 2A and 2B). The 5′-hydroxyl group of the nucleoside analogue was chemically attached via a biodegradable tetraphosphate linker to nanogels using a 2-cyanoethylbis(imidazolyl)phosphate, CNEtOP(O)Im₂, as a phosphorylating reagent. As shown in FIGS. 2A and 2B, the polymers were phosphorylated with CNEtOP(O)Im₂ in DMF and then reacted efficiently with inorganic pyrophosphate in the form of tetra-n-butylammoniumsalt PPi-TBA(6) in order to form the polymeric triphosphate 7. Separately, the 5′-hydroxyl group of the nucleoside was phosphorylated by CNEtOP(O)Im₂, and the activated 5′-phosphorylated nucleoside 8 was reacted in the next step with the polymeric triphosphate 7.

In preliminary experiments, it was found that a similar phosphorylating agent, methyl-bis(imidazolyl) phosphate, MeOPOIm₂, reacted efficiently with the primary hydroxyl groups of nucleosides in the formation of activated 5′-monophosphates, but compared with CNEtOP(O)Im₂, this reaction was much slower. The relative efficacy of phosphorylation using these two reagents was compared based on the yields of nucleoside 5′-monophosphate analyzed by ion-pair HPLC. During the first hour of reaction, CNEtOP(O)Im₂ yielded 80% of monophosphate compared with only 10% when MeOPOIm₂ was used. The electron donor effect of the methoxy group makes the phosphorus atom less electrophilic, which resulted in longer reaction times, especially with secondary hydroxyl groups. However, both phosphorylating agents formed the same final activated 5′-monophosphorylated nucleoside in the instant synthesis. Next, the activated imidazolyl-phosphate moiety in 8 readily reacted with polymertriphosphate 7 and converted into nucleoside 5′-tetraphosphate anchored to CPVA, (9-11) or CDex (14, 15). Initially, thymidine (T) was used as a model drug in this study. Polymeric conjugates were purified by extensive dialysis to remove all reactants. CPVA- and CDex-conjugated 5′-tetraphosphates of FdU were designated as CPVA13-p₄FdU, CPVA31-p₄FdU, CDex9-p₄FdU, and CDex9-p₄T and their properties are shown in Table 1. The amount of nucleoside attached to the polymer was determined by UV absorbance. A high degree of nucleoside loading in nanogels was observed, which was equal to 0.4-0.5 μmol/mg.

TABLE 1
Particle characteristics of polymeric conjugates.

| polymeric | dₕ, nm (volume- | | |
|---|---|---|---|
| conjugate^{a} | averaged) | PDI | ζ, mV |
| CPVA31 | 35.00 ± 1.30 | 0.361 ± 0.01 | 0.00 ± 3.70 |
| CPVA31-p₄FdU | 42.12 ± 6.41 | 0.405 ± 0.04 | −8.47 ± 3.60 |
| CPVA13 | 12.52 ± 5.12 | 0.596 ± 0.03 | −2.57 ± 3.40 |
| CPVA13-p₄FdU | 34.95 ± 5.43 | 0.417 ± 0.01 | −34.0 ± 4.67 |
| CDEX9 | 44.53 ± 8.34 | 0.458 ± 0.02 | −8.00 ± 4.02 |
| CDEX9-p₄FdU | 26.23 ± 4.12 | 0.440 ± 0.03 | −34.80 ± 5.06 |
| CDEX9-p₄T | 18.27 ± 2.01 | 0.508 ± 0.00 | 0.00 ± 4.52 |

^{a}Particle size (dₕ),
polydispersity index (PDI), and
zeta potential (ζ) were measured in 1% solutions in water after 2 hours sonication.
The results are average values ± SD of three measurements.

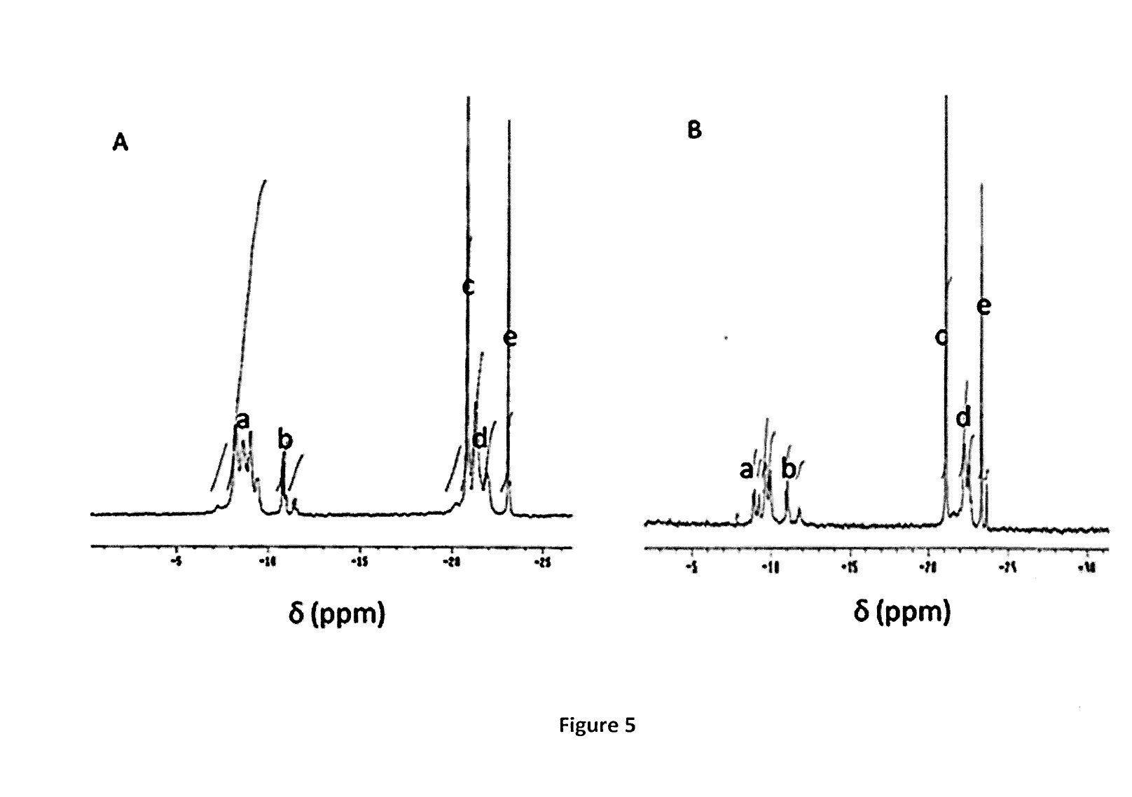

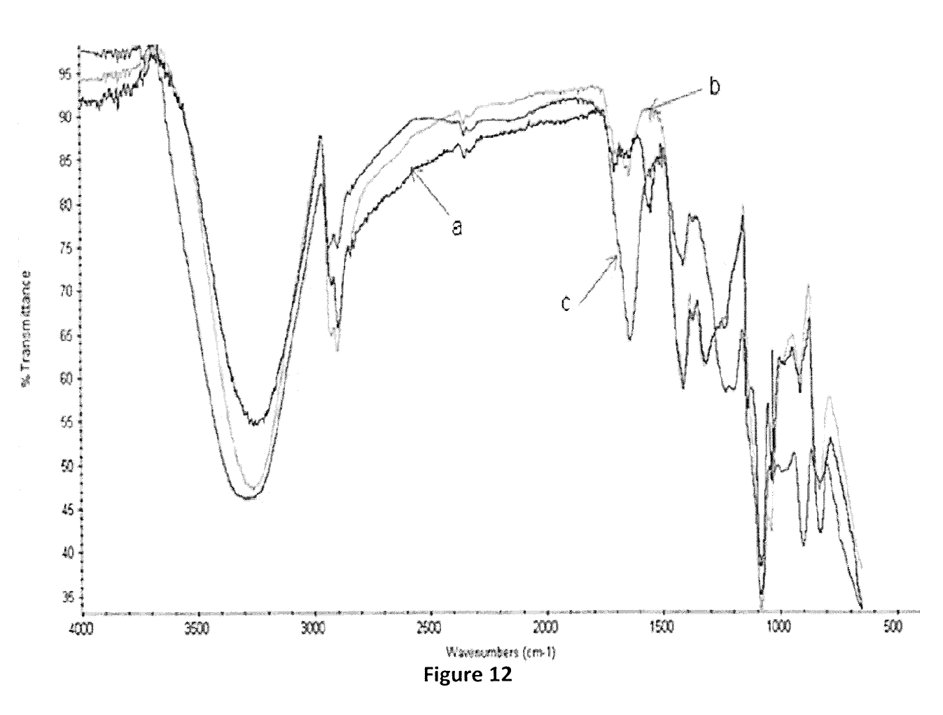

Covalent conjugation of the nucleoside to nanogels via a tetraphosphate linker was further verified by ³¹P NMR and IR spectroscopy. ³¹P NMR spectra confirmed the formation of tetraphosphate structures along with trace amounts of a triphosphate and polyphosphates such as a pentaphosphate (FIG. 5). According to published chemical shifts (Moreno et al. (2000) J. Biol. Chem., 275:28356-28362; Warnecke et al. (2009) J. Org. Chem., 74:3024-3030), upper field region of ³¹P NMR spectra at −20 to −25 ppm corresponds to β- and γ-phosphates (peaks c, d, and e), while signals between −8 and −12 ppm correspond to terminal α- and δ-phosphates (peaks a and b). IR spectra of CPVA-p4FdU allows observation of the conjugation of the FdU nucleoside to the CPVA nanogel (FIG. 12). As is clearly shown in the spectra, the peak at 899 cm⁻¹ corresponding to P—OR stretch, the peak at 1234 cm⁻¹ corresponding to P═O stretch of phosphate, and the peak at 1708 cm⁻¹ corresponding to C═O stretch of amide are clearly increased in the CPVA-p₄FdU products, while peaks at 1311-1417 cm⁻¹ corresponding to O—H bonding of CPVA were reduced, confirming the formation of a polyphosphate linker between CPVA and FdU.

### Particle Size and Zeta-Potential

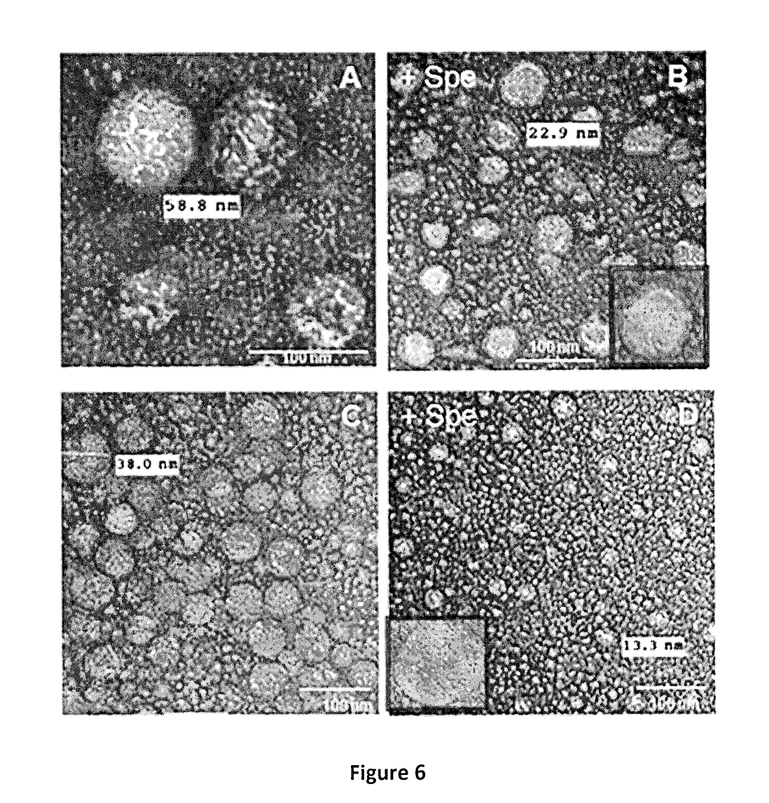

Compact nanogel conjugates could be successfully formed as the result of self-organization of CPVA/CDex-p₄FdU molecules during ultrasonication in aqueous solutions. When sufficient energy was applied, the cholesterol moieties formed compact intramolecular clusters surrounded by a hydrophilic polymeric shell containing the embedded negatively charged drug molecules. The particle size, homogeneity, and morphology of CPVA/CDex-p₄FdU structures were measured by dynamic light scattering (DLS) and transmission electron microscopy (TEM). A single sharp peak in DLS profiles with a hydrodynamic diameter in the range of 12-45 nm implied the presence of a single population of small particles with a relatively low polydispersity index of 0.30-0.59 (Table 1). The high negative zeta-potential of CPVA/CDexp₄FdU confirmed the presence of phosphates in the surface layer of polymeric conjugates. TEM pictures showed the spherical particle morphology with hydrodynamic diameters in the range 38-58 nm (FIG. 6). The change in particle size after neutralization of the negative charge in polymeric conjugates with the polyamine spermine was also studied (FIG. 1). The addition of positively charged spermine at physiological conditions resulted in a 2-3-fold reduction in hydrodynamic and TEM-observed diameters and the change in morphology of particles, e.g., the appearance of a thicker electron-dense exterior layer surrounding these compacted polymeric conjugates (Chen et al. (2005)

### Chemistry 11:4835-4840).

### Enzymatic Hydrolysis

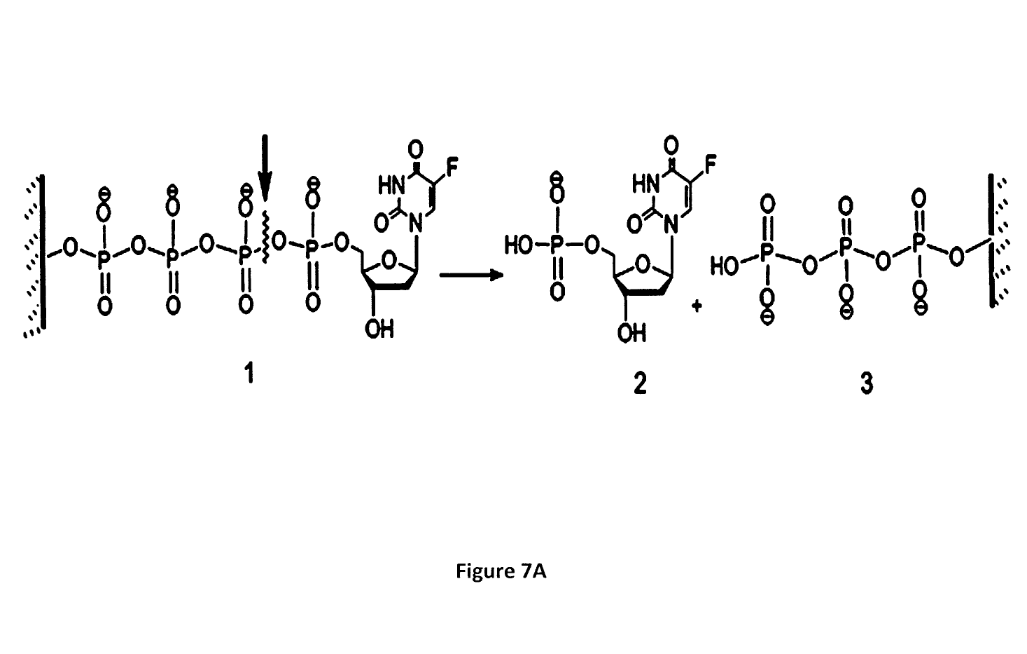

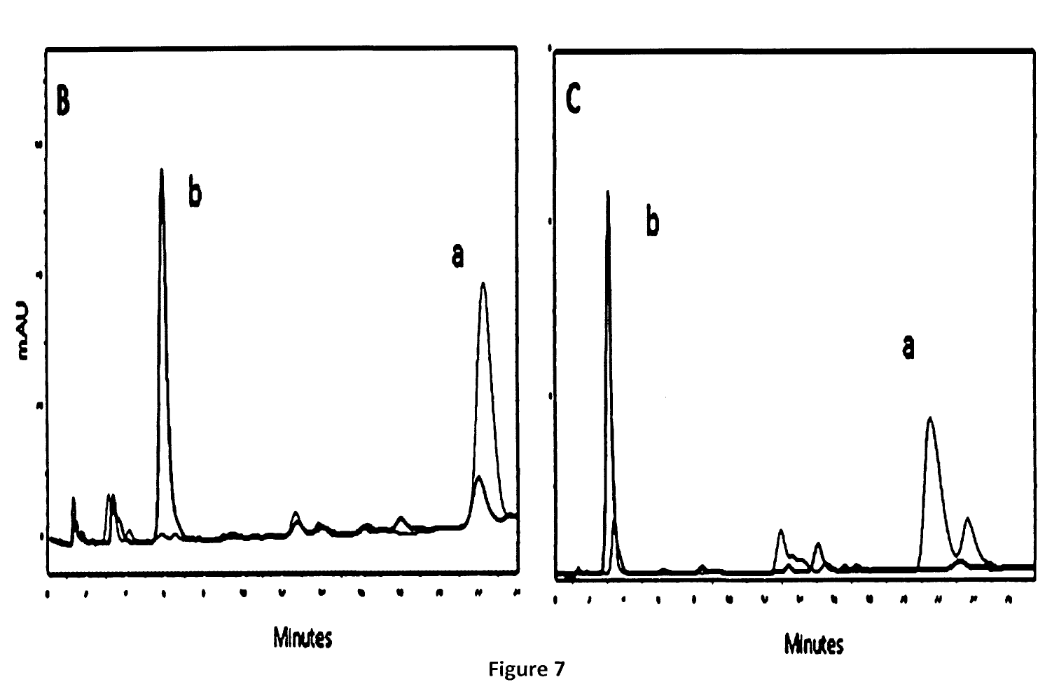

The formation of natural nucleoside 5′-phosphate bonds between nucleoside and nanogel backbone was further confirmed using enzymatic hydrolysis by snake venom phosphodiesterase I (VPDE). VPDE was able to catalyze the hydrolysis of esterified nucleoside 5′-phosphates into a nucleoside 5′-phosphate and also the cleavage of nucleoside 5′-phosphate from oligonucleotides with a free 3′-end (Razzell et al. (1959) J. Biol. Chem., 234:2105-2113; Garcia-Diaz et al. (1991) Eur. J. Biochem., 196:451-457). This property of VPDE allows for the investigation of the enzymatic stability of nanogel-bound nucleoside phosphates. NanogelsCPVA-p₄FdU and CDex-p₄FdU were incubated with VPDE at 37° C. and then analyzed by ion-pair HPLC (FIG. 7). The gradual disappearance of the initial wide peak was observed with the elution time of 25-34 minutes (a) and the formation of a sharp peak at the elution time of ca. 5 minutes (b), which corresponded to control nucleoside 5′-phosphates (TMP or FdUMP). Structure of the released nucleoside products was further confirmed by comparison of UV spectra with initial nucleoside analogues. The data demonstrated a nearly quantitative enzymatic release of nucleotide 5′-phosphate from polymeric conjugates within 12 to 24 hours.

### In Vitro Drug Release

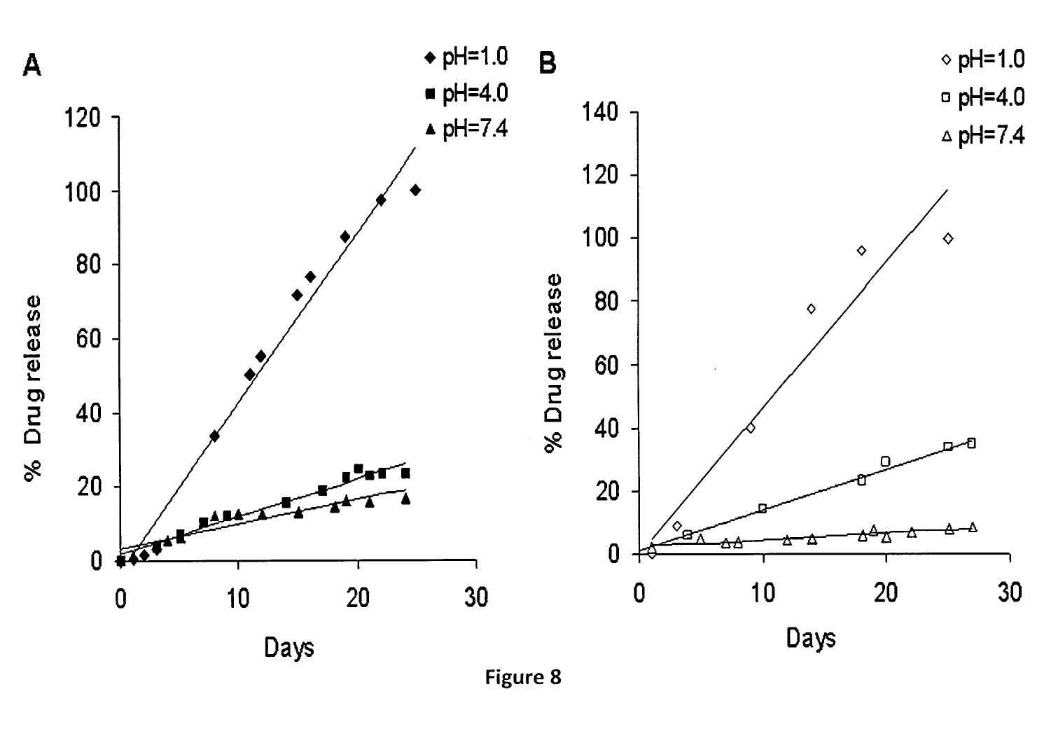

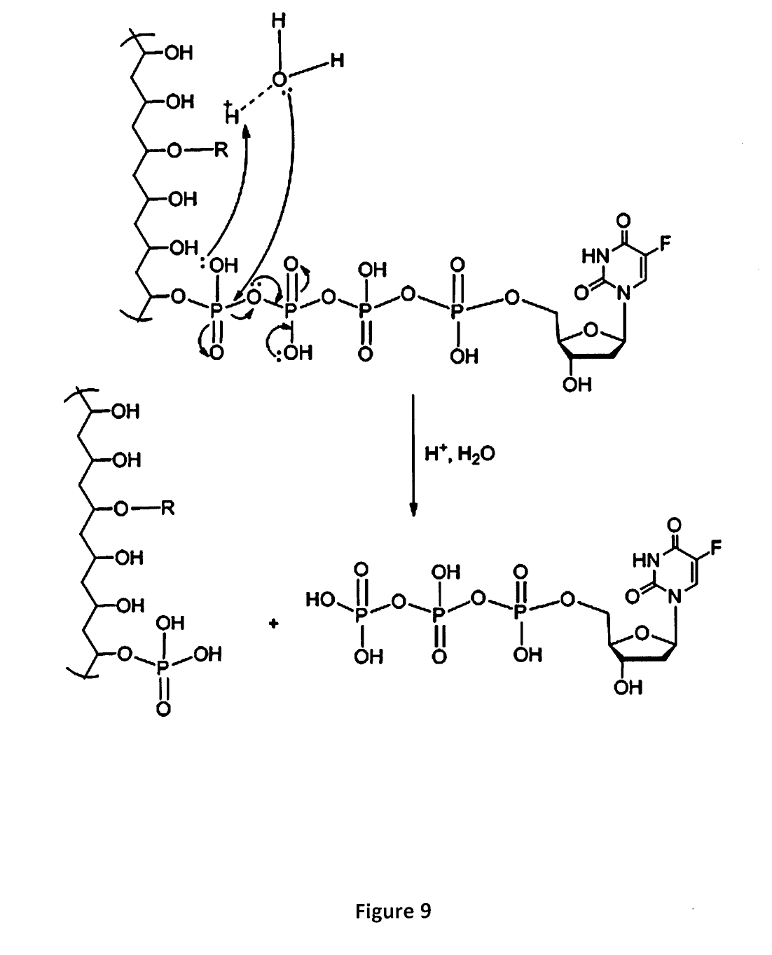

The in vitro release of FdU was monitored at 37° C. at different pH values (1.0, 4.0, and 7.4) in order to assess the stability of nanogel conjugates in the environments in the stomach, endosomal vesicles, and blood, respectively. Each nanogel, CPVA-p₄FdU or CDEX-p₄FdU, was placed into the appropriate buffer solution and incubated at 37° C. Serial aliquots were removed at the appropriate times during hydrolysis, and the cleaved nucleoside/nucleotide was separated by ultrafiltration and quantified by UV absorbance. The release profiles at different pH values are shown in FIG. 8. In general, polymeric conjugates displayed linear first-order reaction kinetics of hydrolysis with slower drug release at pH 7.4 and pH 4.0 than at pH 1.0. At pH 4.0 and 7.4, drug release was 1-2% per day, while at pH 1.0 drug release reached 4% per day. These results are consistent with the pH-dependent degradation behavior of other dinucleoside polyphosphates, such as diadenosine-P1,P3-triphosphate and P1,P4-tetraphosphate (Mikkola, S. (2004) Org. Biomol. Chem., 2:770-776). However, CPVA/CDex-p₄FdU conjugates showed much slower drug release compared to diadenosine-P1,P4-tetraphosphate (25 days vs 3 days). The pH-dependent hydrolysis can be facilitated by a hydronium ion through the nucleophilic attack of protonated phosphate groups at lower pH, which results in an S_{N}(P)-type substitution, and then the formation of a penta-coordinated phosphorus transition state and nucleoside 5′-phosphates as final products (FIG. 9). Highly hydrated polymer coils surrounding the tetraphosphate linkers evidently create a steric hindrance and significantly slow down the process.

Evidently, the release of phosphorylated nucleosides in cancer cells would provide a strong therapeutic advantage to these polymeric conjugates, because the drug component does not have to pass through the phosphorylation step, which is known to be a rate-limiting step in biological activation of nucleoside analogues (Longley et al. (2003) Nat. Rev. Cancer 3:330-338). The data show that this type of polymeric conjugates is capable of the sustained drug release during the extended period of time and can serve as active drug depot, significantly enhancing therapeutic effect against cancer cells. Peritumoral injections or systemic administration of polymeric conjugates would result in only minimal initial drug burst, a shortcoming of many drug delivery systems at systemic administration. Additionally, accumulation of nanocarriers from blood circulation through the enhanced permeability and retention (EPR) effect in leaking tumor neovasculature can potentially enhance the tumor growth inhibitory effect. Polymeric conjugates can also be considered as potential oral therapeutic formulations due to the observed slow drug release at low pH in the digestive tract (in vivo experiments are underway). It has also been demonstrated that enzymatic hydrolysis of polymeric conjugates is at least 20-25 times faster than hydrolytic hydrolysis. Therefore, these polymeric conjugates, which have a slow, sustained drug release in tumor tissue and other organs in normal conditions in vivo, might be quickly activated by enzymatic activities present in the cytosol or subcellular compartments of proliferating cancer cells (Bender et al. (2006) Pharmacol. Rev., 58:488-520). The most common type of hydrolytic enzymes in mammalian cells are cytosolic phosphodiesterases and nucleotide phosphatases, which can release active 5′-nucleotides from the polymeric conjugates (Garcia-Diaz et al. (2006) Eur. J. Biochem., 196:451-457).

### Cytotoxicity Assay

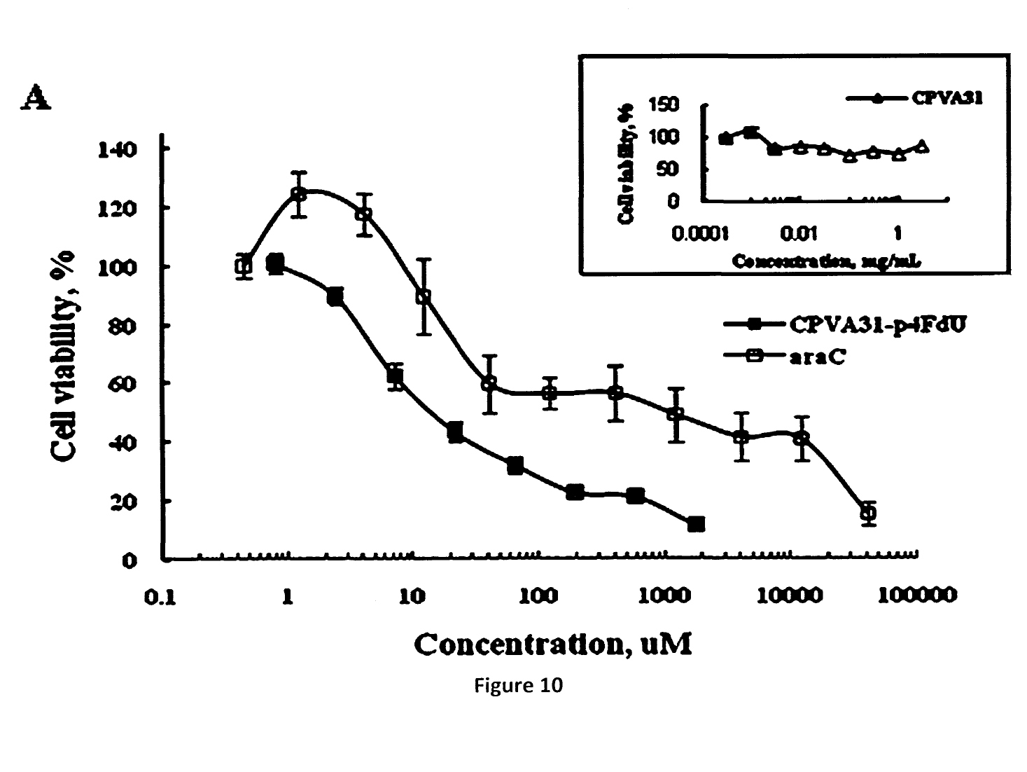

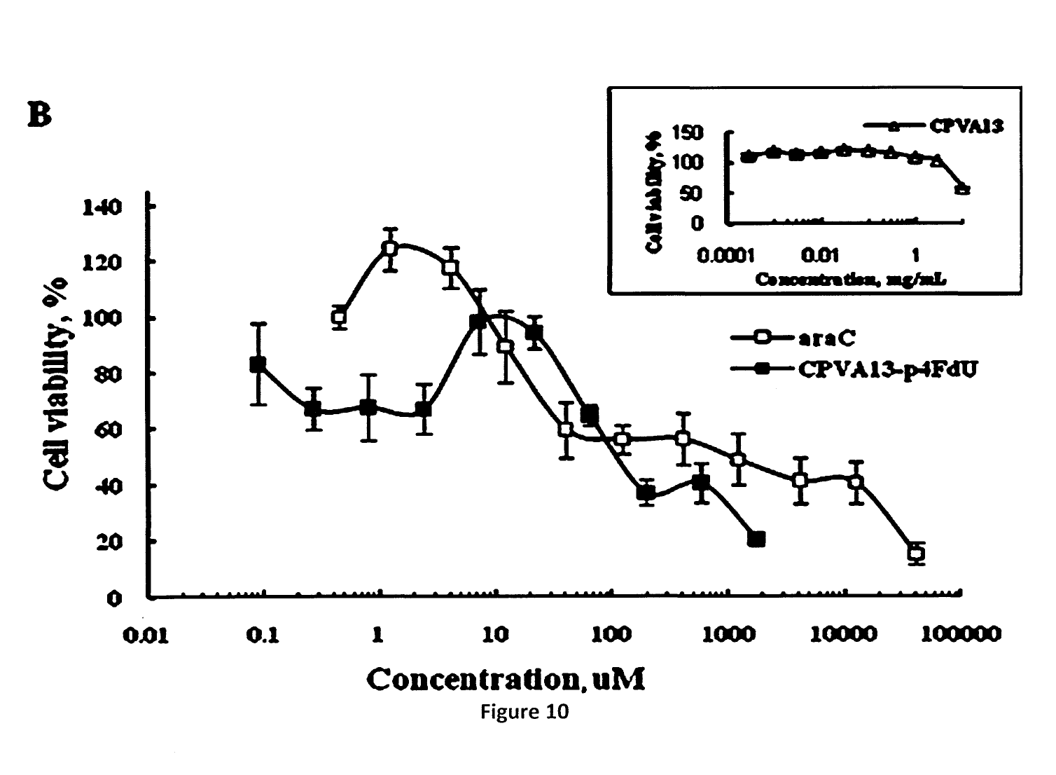

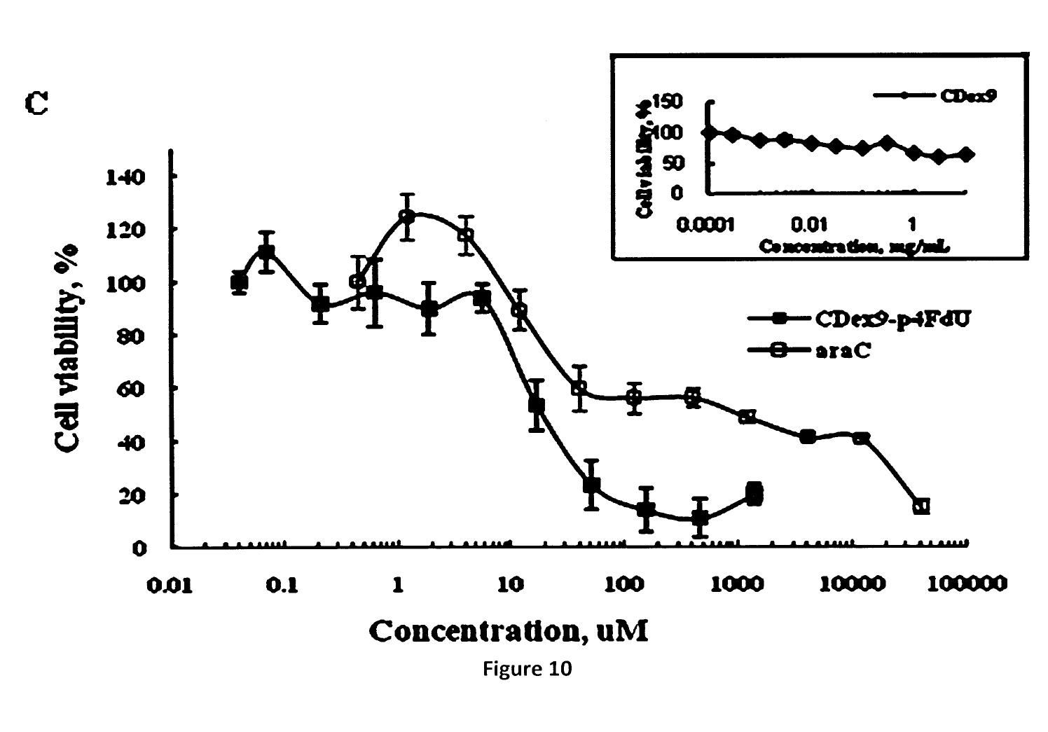

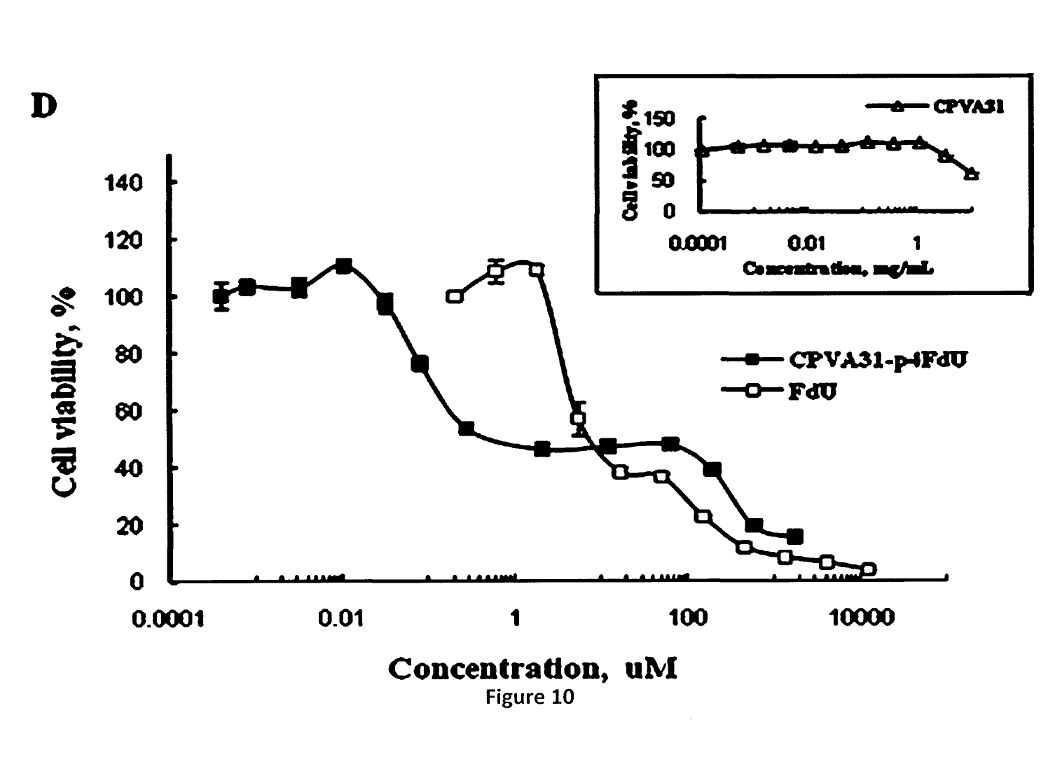

Drug resistance to nucleoside analogues is known to be an important clinical problem in the treatment of cancer. Therapeutic effects can be achieved with nucleoside analogues as a single agent or in combination with other drugs only at the increasingly higher dosage. Here, the cytotoxicity of several polymeric conjugates of activated analogues of 5-fluorouracyl were studied as a model drug in various cancer cell lines including ones that are resistant to nucleoside analogues. The cytotoxicity of polymeric floxuridine conjugates CPVA13-p₄FdU, CPVA31-p₄FdU, and CDEX9-p₄FdU was determined in human prostate adenocarcinoma PC-3, breast carcinoma MCF-7 and MDA-MB-231, hepatic carcinoma HepG2, gemcitabine-resistant follicular lymphoma RL7/G, and cytarabine-resistant T-lymphoma CEM/araC/8 cells using a thiazolyl blue (MTT) dye reduction assay (Hansen et al. (1989) J. Immunol. Methods 119:203-210). As shown in FIG. 10 and Table 2, nanoconjugates showed considerably enhanced cytotoxicity and lower IC₅₀ values (drug concentration resulting in 50% cell death) compared to free floxuridine in all of these cell lines. The enhancement factor (EF), which is equal to IC₅₀ (drug)/IC₅₀ (conjugate), was used as a measure of the increase in cytotoxicity of polymeric conjugates compared to free drug. Nanogels without conjugated drug demonstrated no cytotoxicity (IC₅₀>10 mg/mL). All of these polymeric conjugates showed a higher EF in drug resistant cell lines, CEM/araC/8 and RL7/G, compared to other tumor cells. Specifically, CPVA13-p₄FdU and CDex9-p₄FdU exhibited an EF of 100 and 85 in CEM/araC/8 cells, while the EF showed by other cells was normally in the lower range of 3.5-50.

TABLE 2
Cytotoxicity of cytotoxic drugs, polymeric conjugates,
and drug conjugates in cancer cells.

| | IC₅₀ values (μM)^{a} | | | | | |
|---|---|---|---|---|---|---|
| | | | | MDA- | CEM/ | |
| Drug Formulation | PC-3 | MCF-7 | Hep G2 | MB-231 | araC/8 | RL7/G |
| Floxuridine (FdU) | 6.5 | 12195 | 2195 | 14.2 | | |
| Cytarabine (araC) | | | | | 1234 | |
| Gemcitabine (G) | | | | | | 19011 |
| CPVA31 | >10 | >10 | >10 | >10 | >10 | >10 |
| CPVA13 | >10 | >10 | >10 | >10 | >10 | >10 |
| CDEX9 | | | | | >10 | >10 |
| CPVA31-p₄FdU | 0.4 | 2032 | 487 | 0.28 | 12.3 | 3802 |
| | EF = 16 | EF = 6 | EF = 4.5 | EF = 50 | EF = 100 | EF = 5 |
| CPVA13-p₄FdU | 81.3 | 813 | 609.7 | 0.6 | 102 | 950 |
| | | EF = 15 | EF = 3.6 | EF = 23 | EF = 12 | EF = 20 |
| CDEX9-p₄FdU | | | | | 14.5 | |
| | | | | | EF = 85 | |

^{a}Cytotoxicity was measured after 72-hour treatment. IC₅₀ of CPVA31, CPVA13, and CDEX9 in each cell line are given in mg/mL. EF = enhancement factor showing the efficacy compared to free drug.

### In Vivo Tumor Growth Inhibition

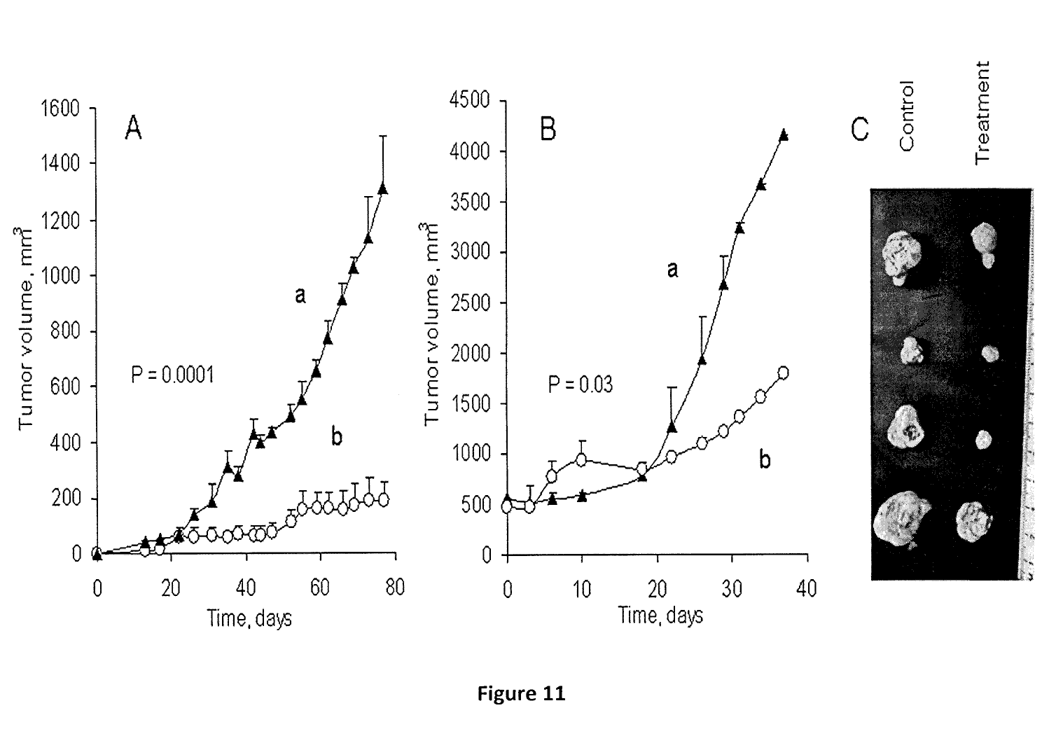

The therapeutic efficacy of polymeric conjugates was evaluated in subcutaneous (s.c.) human prostate adenocarcinoma PC-3 and gemcitabine-resistant follicular lymphoma RL7/G tumor xenograft mouse models. These tumors were established by s.c. injection of tumor cells in the lower flank areas of athymic nu-nu mice. After the observation of the initial tumors, animals were randomly separated into control and treatment groups (n=5-6). Median tumor volume was measured by digital calipers twice a week simultaneously with peritumoral injections of polymeric conjugates with a dose of 80 mg/kg, which is equivalent to 10 mg FdU/kg. This way of administration allowed a subcutaneous storage of the viscous polymer-drug conjugate and the sustained release of activated drug into the tumor. A strong 6.5-fold tumor growth inhibition in human prostate carcinoma PC-3 model was observed that confirms the high therapeutic potential of polymeric conjugates (FIG. 11A). Although the observed 2-fold growth inhibition was lower in drug-resistant human follicular lymphoma RL7/G tumors compared to PC-3 tumors, the effect of the CPVA31-p₄FdU conjugate was statistically significant (P<0.05) (FIG. 11B). The significance of the experimental data was determined by a two-tailed Student's t-test. No significant weight loss or acute toxicity of polymeric conjugates was observed during the entire period of treatment. Tumors removed from experimental animals at the end of the experiments were clearly smaller than tumors in the control group (FIG. 11C).

Thus, a novel type of covalently bound polymeric phosphorylated nucleoside analogues, polymeric conjugates has been developed for a sustained delivery of the activated anticancer drugs into tumors. These carriers combine attractive properties of biocompatible polymers with an enhanced cytotoxic efficacy of activated nucleoside analogues and form compact drug-loaded polymeric nanoparticles. In vitro evaluation against various cancer cell lines, including drug-resistant cancer cells, demonstrated that the activated floxuridine conjugate has 50-100 times stronger cytotoxicity compared to free nucleoside analogue. Furthermore, the observed sustained drug release was may lead to the observed increase in tumor growth inhibition following the peritumoral administration of polymeric conjugates in subcutaneous tumor xenograft models. This class of anticancer drug formulations also has other features which makes them a vehicle for oral administration of activated phosphorylated nucleoside analogues.

### Example 2

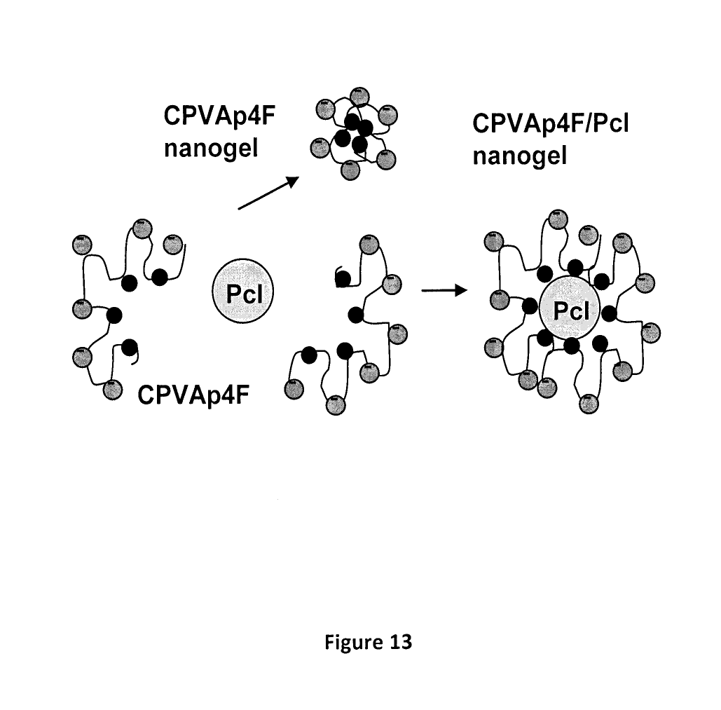

FIG. 13 provides a schematic of the formation of the dual drug formulations of drug-nanogel conjugates. Specifically, paclitaxel (Pcl) and 17-N-allylamino-17-demethoxygeldanamycin (17-AAG) were each stabilized within nanogel formulations. Dynamic light scattering showed a narrow size distribution with an average diameter of about 300 nm for the dual drug formulations.

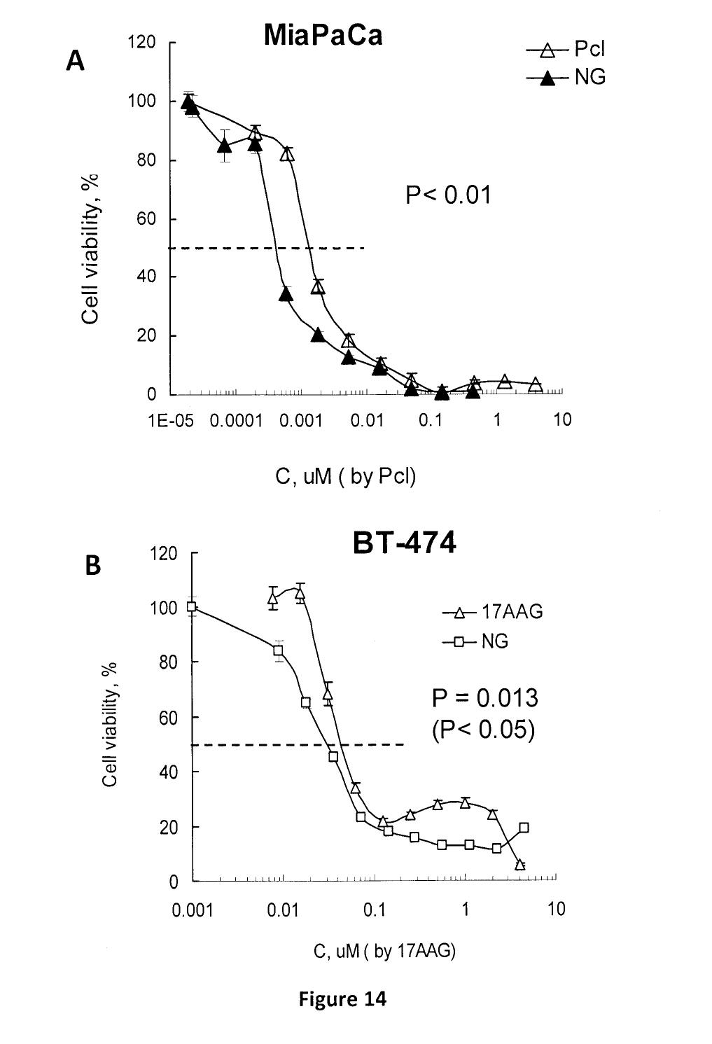

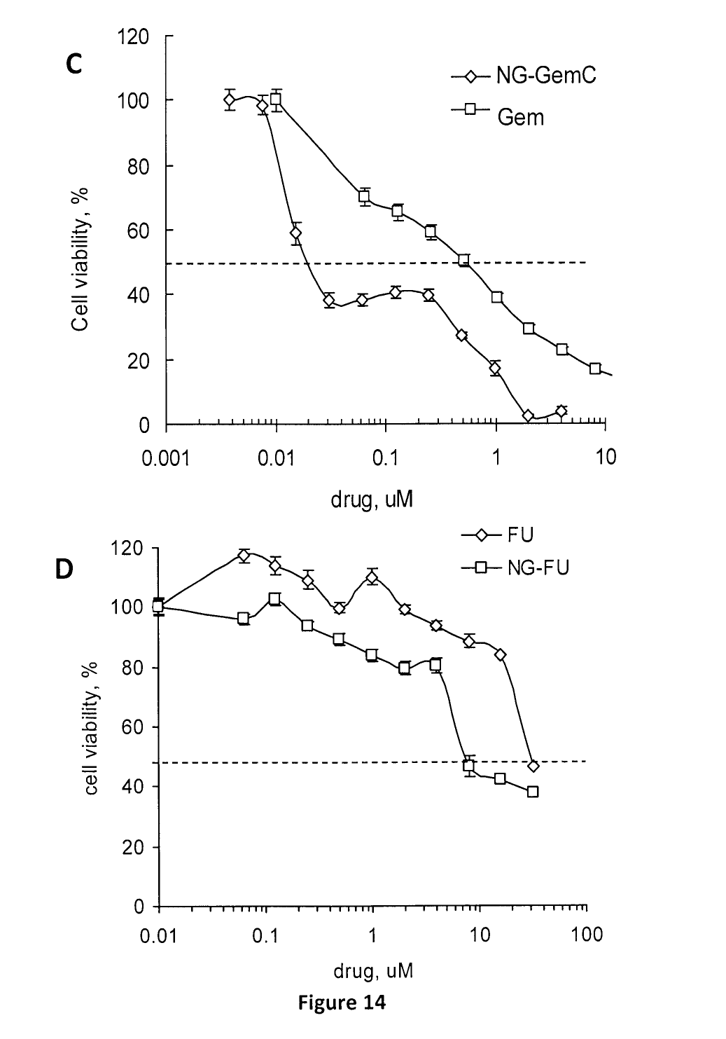

The cytotoxicity of the dual drug nanoformulations was analyzed in different cancer cell lines by a standard MTT assay. FIGS. 14A and 14B show that the dual drug nanoformulations of CPVAp4FdU and paclitaxel and CPVAp4FdU and 17-AAG, respectively, showed synergistic effects against a pancreatic cancer (MIA PaCa) and breast cancer (BT-474) cell line when compared to paclitaxel and 17-AAG alone. FIG. 14C also demonstrates that the dual drug nanoformulation of CPVAp4FdU and gemcitabine showed synergistic effects against a pancreatic cancer cell line (MIA PaCa) when compared to gemcitabine alone. FIG. 14D demonstrates that CPVAp4FdU was more effective against a breast cancer cell line (BT-474) when compared to FdU alone.

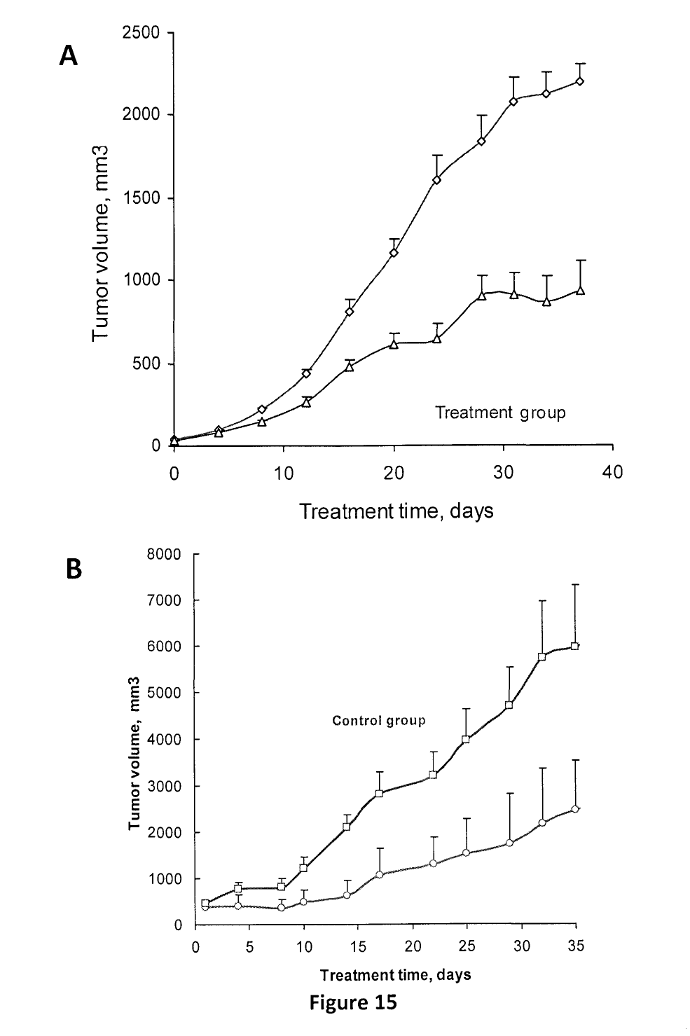

The therapeutic efficacy of the dual drug nanoformulation was evaluated in subcutaneous (s.c.) gemcitabine-resistant follicular lymphoma RL7/G tumor xenograft mouse model. These tumors were established by s.c. injection of tumor cells in the lower flank areas of athymic nu-nu mice. After the observation of the initial tumors, animals were randomly separated into control and treatment groups (n=7-8). Median tumor volume was measured by digital calipers twice a week. The dual drug nanoformulation of CPVAp4FdU and gemcitabine was administered by oral gavage as aqueous solution (2 mg/0.5 ml) once every three days. As seen in FIG. 15A, the dual drug nanoformulations led a significant reduction in tumor volume (P<0.001).

The therapeutic efficacy of CPVAp4FdU was also evaluated in subcutaneous (s.c.) gemcitabine-resistant follicular lymphoma RL7/G tumor xenograft mouse model. These tumors were established by s.c. injection of tumor cells in the lower flank areas of athymic nu-nu mice. After the observation of the initial tumors, animals were randomly separated into control and treatment groups (n=5). Median tumor volume was measured by digital calipers twice a week. CPVAp4FdU was administered by oral ad libitum of an aqueous solution (0.16 mg/ml). As seen in FIG. 15B, the oral delivery of CPVAp4FdU led a significant reduction in tumor volume (P<0.01).

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A nanoparticle comprising
a) a hydrophilic polymer conjugated to hydrophobic moieties; and
b) a therapeutic agent conjugated to said hydrophilic polymer by a polyphosphate linkage,
wherein said hydrophobic moieties form a hydrophobic core of said nanoparticle.

2. The nanoparticle of claim 1, wherein said polyphosphate linkage is a tetraphosphate.

3. The nanoparticle of claim 1, wherein said therapeutic agent is a nucleoside analog.

4. The nanoparticle of claim 3, wherein said nucleoside analog is selected from the group consisting of floxuridine, azidothymidine, abacavir, fludarabine, 5-fluorouracil, and gemcitabine.

5. The nanoparticle of claim 1, wherein said hydrophobic moiety is selected from the group consisting of lipid, fatty acid, steroid, and cholesterol.

6. The nanoparticle of claim 1, wherein said polymer comprises hydroxyl groups.

7. The nanoparticle of claim 1, wherein said polymer is selected from the group consisting of polyvinyl alcohol, polysaccharide, dextran, dextrin, pectin, chitin, chitosan, hyaluronic acid, and copolymers thereof.

8. The nanoparticle of claim 1, wherein said nanoparticle is linked to at least one targeting ligand.

9. The nanoparticle of claim 1, wherein said nanoparticle encapsulates at least one therapeutic agent.

10. A composition comprising at least one nanoparticle of claim 1 and at least one pharmaceutically acceptable carrier.

11. A method for treating or inhibiting a disease or disorder in a subject in need thereof, said method comprising administering to said subject at least one nanoparticle of claim 1.

12. The method of claim 11, wherein said disease or disorder is cancer.

13. The method of claim 12, wherein said cancer is a drug resistant cancer.

14. The method of claim 11, wherein said disease or disorder is viral infection.

15. A method of synthesizing the nanoparticle of claim 1, said method comprising:
a) conjugating hydrophobic moieties to a hydrophilic polymer;
b) conjugating a polyphosphate linker to the hydrophilic polymer; and
c) conjugating a therapeutic agent to said polyphosphate linker.

16. The method of claim 15 further comprising sonicating the nanoparticles in an aqueous solution.
